(19) United States
(12) Patent Application Publication
(10) Pub. No.: US 2013/0136727 A1
(43) Pub. Date: May 30, 2013

(54) Angiogenin and Variants Thereof for Treatment of Neurodegenerative Diseases

(71) Applicants: Guo-fu Hu, Wellesley, MA (US); Hiroko Kishikawa, Brighton, MA (US)

(72) Inventors: Guo-fu Hu, Wellesley, MA (US); Hiroko Kishikawa, Brighton, MA (US)

(73) Assignee: PRESIDENT AND FELLOWS OF HARVARD COLLEGE, Cambridge, MA (US)

(21) Appl. No.: 13/510,675

(22) PCT Filed: Nov. 3, 2010
(86) PCT No.: PCT/US2010/055233

§ 371 Date: Sep. 5, 2012

Related U.S. Application Data
(60) Provisional application No. 61/262,679, filed on Nov. 19, 2009.

(51) Int. Cl. C12N 9/22

(52) U.S. Cl. CPC C12N 9/22
USPC 424/94.6; 435/199; 514/44 R

(57) ABSTRACT

Methods and compositions for treating neurodegenerative disorders using angiogenin and/or angiogenin variants are provided.

### RELATED APPLICATIONS

This application claims priority to U.S. Provisional Application No. 61/262,679, filed on Nov. 19, 2009 and is hereby incorporated herein by reference in its entirety for all purposes.

### STATEMENT OF GOVERNMENT INTERESTS

This invention was made with government support under the National Institutes of Health grant number CA105241. The Government has certain rights in the invention.

### FIELD

The present invention relates to methods and compositions for treating neurodegenerative diseases and disorders such as, for example, amyotrophic lateral sclerosis.

### BACKGROUND

Amyotrophic lateral sclerosis (ALS) is a progressive neurodegenerative disease with specific loss of motor neurons in the brain, brain stem, and spinal cord (Pasinelli and Brown (2006) Nat. Rev. Neurosci. 7:710). The average age of onset is 55 years with upper and lower motor neuron signs, including distal muscle weakness and wasting, increased muscle tone with hyperreflexia, and at times diaphragmatic and/or bulbar weakness. Atypical forms can include symptoms of dementia and/or Parkinsonism. Both familial and sporadic forms of the disease have been reported and described as having no distinguishable differences in symptoms, progression, and histological abnormalities. All forms of ALS progress to generalized amyotrophy, culminating in respiratory failure and death after an average duration of four years (Rowland and Shneider (2001) N. Engl. J. Med. 344:1688).

ALS was first referred to as Charcot's sclerosis in honor of the French neurobiologist Jean-Martin Charcot who originally described this disease in 1869 (Charcot and Joffory (1869) Arch. Physiol. Neurol. Pathol. 2:744). It is now more commonly known as Lou Gehrig's disease in the United States, in memory of the great baseball player who developed the disease in the 1930s. It is a devastating disease as there is almost no cognitive impairment at a time when nearly complete paralysis of arms, legs, and the muscles necessary for speech has developed (Rippon et al. (2006) Arch. Neurol. 63:345).

Since 2004, angiogenin (ANG), a 14 kDa angiogenic ribonuclease, has emerged as an important gene and/or genetic factor in ALS (Greenway et al. (2006) Nat. Genet. 38:411). A total of 15 different missense mutations in the coding region of ANG have been identified in 33 of 3001 patients from Irish, Scottish, Swedish, North American, French, and Italian ALS populations (Greenway et al. (2006) Nat. Genet. 38:411; Conforti et al. (2007) Neuromuscul. Disord.; Gellera et al. (2007) Neurogenetics; Wu et al. (2007) Ann. Neurol. 62:609; Paube et al. (2008) 65:1333), which places ANG as the third most frequently mutated gene in ALS (after SOD1 and TARDBP). Importantly, while wild type (WT) ANG induces angiogenesis, stimulates neurite outgrowth of motor neurons and protects them from hypoxia-induced death, mutant ANG proteins lack these activities (Wu et al. (2007) Ann. Neurol. 62:609; Subramanian et al. (2008) Hum. Mol. Genet. 17:130). Therefore, ANG appears to be the first loss-of-function gene so far identified in ALS (Wu et al. (2007) Ann. Neurol. 62:609). The genes and genetic factors whose alterations predispose to ALS are listed below.

Genes and genetic factors associated with ALS are as follows (disease type/locus/gene/inheritance/clinical features/reference): ALS1/21q22.21/SOD1/autosomal dominant (AD)/adult onset/typical ALS/(Rosen et al. (1993) Nature 362:59); ALS2/2q33/Alsin/autosomal recessive (AR)/juvenile onset, atypical ALS and primary lateral sclerosis (PLS), slow progression/(Yang et al. (2001) Nat. Genet. 29:160); ALS3/18q21/unknown/AD/adult onset, typical ALS/(Hand et al. (2002) Am. J. Hum. Genet. 70:251); ALS4/9q34/SETX/AD/juvenile onset, atypical ALS, slow progression/(Chen et al. (2004) Am. J. Hum. Genet. 74:1128); ALS5/15q15.1-21.1/unknown/AR/juvenile onset, atypical ALS, slow progression, no pseudobulbar signs/(Hentati et al. (1998) Neurogenetics 2:55); ALS6/16q12/unknown/AD/adult onset, typical ALS, short duration/(Abalkhail et al. (2003) Am. J. Hum. Genet. 73:383); ALS7/20ptel-13/unknown AD/adult onset, typical ALS, short duration/(Sapp et al. (2003) Am. J. Hum. Genet. 73:397); ALS8/20q13.33/VAPB (vesicle-associated membrane protein, a 27.2 kDa homodimer belonging to a family of intracellular vesicle-associated/membrane-bound proteins that are presumed to regulate vesicle transport)/AD/adult onset, atypical ALS, slow progression/(Nishimura et al. (2004) Am. J. Hum. Genet. 75:822); ALS-frontotemporal dementia (FTD)/9q21-22/unknown/AD/adult onset, ALS with FTD/(Hosler et al. (2000) JAMA 284:1664); ALS-PD/17q21/MAPT (microtubule-associated protein Tau)/AD/adult onset, ALS with Parkinsonism and dementia/(Hutton et al. (1998) Nature 393:702); progressive lower MND/2p13/DCTN1 (dynactin p150 subunit, a major component of the dynein complex that comprises the major zonal retrograde motor (a mutation in the p150 subunit appears to affect the binding of the dynactin-dynein motor to microtubules))/AD/adult onset, lower motor neuron disorder/(Puls et al. (2003) Nat. Genet. 33:455); ALS, sporadic/6q12, 22q12.1-q13.1, 6q21.3/VEGF (vascular endothelial growth factor, an essential angiogenic factor that also regulates neurogenesis); ATFHC (neurofilament heavy chain); HFE (a hemochromatosis gene involved in iron metabolism)/risk factors/adult onset, typical ALS/(Lambrechts et al. (2003) Nat. Genet. 34:383, Al-Chalabi et al. (1999) Hum. Mol. Genet. 8:157, Figlewicz et al. (1994) Hum. Mol. Genet. 3:1757, Tomkins et al. (1998) Neuroreport. 9:3967, Goodall et al (2005) Neurology 65:934); ALS, familiar and sporadic/14q11.2/ANG/AD/adult onset, typical ALS (Greenway et al. (2006) Nat. Genet. 38:411, Gellera et al. (2007) Neurogenetics, Wu et al. (2007) Ann. Neurol. 62:609). ALS, familiar and sporadic/1p36/TARDBP/AD/adult onset, typical ALS (Gitcho et al. (2008) Ann. Neurol. 63:535; Kabashi et al. (2008) Nat. Genet. 40:572; Sreedharan et al. (2008) Science 319:1668; Van Deerlin (2008) Lancet Neurol. 7:409; Yokoseki et al. (2008) Ann. Neurol. 63:538; Rutherford et al. (20080) PloS Genet. 4:e1000193).

The etiology of ALS is likely to be multi-factorial, involving the interplay of several mechanisms to initiate disease and propagate the spread of motor neuron death. A generally accepted hypothesis at present is that multiple factors, both genetic and environmental, cause mitochondrial dysfunction and excitotoxicity, lead to abnormal protein precipitation, and finally cell apoptosis (Goodall and Morrison (2006) Expert Rev. Mol. Med. 8:1).

There is presently no effective pharmacologic treatment for ALS to halt neuronal death or even slow it appreciably. Riluzole, the only drug approved for ALS since 1995, only extends survival by 2-3 months if it is taken for 18 months. Riluzole is thought to act in part by limiting glutamate release. It preferentially blocks tetrodotoxin sensitive sodium channels, which are associated with damaged neurons (Song et al. (1997) J. Pharmacol. Exp. Ther. 282:707). This reduces influx of calcium ions and indirectly prevents stimulation of glutamate receptors. Together with direct glutamate receptor blockade, the effect of the neurotransmitter glutamate on motor neurons is greatly reduced. Riluzole was approved for use in ALS after two independent clinical trials showed a marginal increase in the survival time of ALS patients (Bensimon et al. (1994) N. Engl. J. Med. 330:585; Lacomblez et al. (1996) Lancet 347:1425). Unfortunately, patients taking riluzole do not experience any slowing in disease progression or improvement in muscle function. Therefore, riluzole does not present a cure, or even an effective treatment. Angiogenin is disclosed to be considered as a treatment for neurodegenerative diseases. See US 2008/0045456. However, the search for therapeutic agents continues.

### SUMMARY

Embodiments of the present invention are directed to the use of angiogenin and angiogenin variants to treat a disease or condition characterized by neuronal injury or death or axonal degeneration, for example neurodegenerative diseases such as Parkinson's disease or Alzheimer's disease or motor neuron diseases such as ALS, primary lateral sclerosis or spinal muscular atrophy. The present invention is based in part on the surprising discovery that ANG protein and variants thereof improve one or more physical characteristics of a neurodegenerative disorder such as, e.g., ALS. For example, it has been surprisingly discovered that i.p. injection of ANG protein improved motor muscular function by 15-fold and increased life span of SOD1^{G93A} mice by 26%. The present invention is also based in part on the surprising discovery that variant ANG proteins improved motor muscular function, increased life span and decreased weight loss in excess of that of angiogenin. For purposes of the present invention, it is to be understood that the teachings herein as they pertain to angiogenin equally apply to the angiogenin variants, or modifications or neuroprotective fragments thereof of the present invention, including treatment results, dosing regimens, delivery vehicles and therapeutic effect.

Embodiments of the present invention include variants of angiogenin and neuroprotective fragments thereof. In particular variants of angiogenin include those that have been modified to increase nuclear translocation and/or ribonucleolytic activity of the angiogenin variant compared to the wild type. Embodiments of the invention include angiogenin where one or more amino acids of the nuclear localization sequence has been changed. As shown in FIG. 19, the nuclear localization sequence of angiogenin is ³⁰MRRRG³⁴ and according to the present invention, one or more of the amino acids M-R-R-R-G are changed to a different amino acid such that the angiogenin variant exhibits increased ability to enter and be retained in the nucleus of a cell. According to certain embodiments, the nuclear localization sequence of wild type angiogenin is ³⁰MRRRG³⁴ and modifications within the scope of the present invention include ³⁰KRRRG³⁴ (M30K), ³⁰RRRRG³⁴ (M30R), ³⁰MRRRK³⁴ (G34K), ³⁰MRRRR³⁴ (G34R), ³⁰KRRRR³⁴ (M30KG34R), ³⁰RRRRK³⁴ (M30RG34K), ³⁰RRRRR³⁴ (M30RG34R), and ³⁰KRRRK³⁴ (M30KG34K). These modifications can occur alone or along with D116H or Q117G substitutions that increase the ribonucleotytic activity of angiogenin or other substitutions that increase the ribonucleotytic activity of angiogenin.

Accordingly, a method of therapeutically treating a neurodegenerative disorder (e.g., ALS and/or spinal muscular atrophy) in a subject in need thereof is provided by administering angiogenin or an angiogenin variant or modification. In certain exemplary embodiments, the method includes administering (e.g., intravenously administering, intramuscularly administering, subcutaneously administering, intraperitoneally administering, intrathecally administrating and/or intraventricularly administrating) to the subject a therapeutically effective amount of a composition including an isolated angiogenin polypeptide, allowing the isolated angiogenin polypeptide to pass through one or both of the blood brain barrier and the blood spinal cord barrier, and reducing one or more symptoms of the neurodegenerative disorder in the subject such that the neurodegenerative disorder is therapeutically treated. In other exemplary embodiments, the method includes administering (e.g., intravenously administering, intramuscularly administering, subcutaneously administering, intraperitoneally administering, intrathecally administrating and/or intraventricularly administrating) to the subject a therapeutically effective amount of a composition including an isolated nucleic acid sequence encoding an angiogenin polypeptide, expressing the angiogenin polypeptide in the subject, allowing the angiogenin polypeptide to pass through one or both of the blood brain barrier and the blood spinal cord barrier, and reducing one or more symptoms of the neurodegenerative disorder in the subject such that the neurodegenerative disorder is therapeutically treated. In still other exemplary embodiments, the method includes administering (e.g., intraventricularly administering and/or intrathecally administering) directly to the central nervous system (CNS) of a subject (using, e.g., an infusion pump and/or a delivery scaffold) a therapeutically effective amount of a composition including an isolated angiogenin polypeptide or a therapeutically effective amount of a composition including an isolated nucleic acid sequence encoding an angiogenin polypeptide, expressing the angiogenin polypeptide in the subject, and reducing one or more symptoms of the neurodegenerative disorder in the subject such that the neurodegenerative disorder is therapeutically treated.

In certain aspects, a method includes one or any combination of the following steps: allowing nuclear translocation of the isolated angiogenin polypeptide; allowing the isolated angiogenin polypeptide to stimulate ribosomal RNA transcription; allowing the isolated angiogenin polypeptide to stimulate ribosomal biogenesis; allowing the isolated angiogenin polypeptide to stimulate cell (e.g., a spinal cord cell and/or one or both of a neural cell (e.g., a motor neuron) and an endothelial cell) proliferation; allowing the isolated angiogenin polypeptide to stimulate cell differentiation (e.g., an undifferentiated cell is stimulated to differentiate into a neural cell); and/or allowing the isolated angiogenin polypeptide to stimulate angiogenesis. In certain aspects, a method includes allowing nuclear translocation of the isolated angiogenin polypeptide, allowing the isolated angiogenin polypeptide to stimulate ribosomal RNA transcription, allowing ribosomal biogenesis, allowing cell proliferation and allowing angiogenesis. In certain aspects, a method includes allowing the isolated angiogenin polypeptide to stimulate angiogenesis in the CNS of a subject.

In certain aspects, one or more symptoms of ALS, e.g., motor neuron degeneration, muscle weakness, muscle atrophy, fasciculation development, frontotemporal dementia and/or premature death are improved in the subject. In certain aspects, the angiogenin polypeptide enters one or both of the brain and the spinal cord. In other aspects, one or both of muscle coordination and muscle function are improved. In other aspects, the survival of the subject is prolonged. In other aspects, weight loss is decreased. In still other aspects, a nucleic acid sequence is administered using a gene therapy vector.

In certain exemplary embodiments, ANG mutant polypeptides are provided. In certain aspects, a mutant ANG polypeptide is administered to a subject or a cell to regain and/or enhance one or more ANG functions in the subject or cell.

In certain exemplary embodiments, a method of increasing one or more ANG activities in a subject including administering to the subject a composition comprising an isolated angiogenin polypeptide having at least one mutation, and allowing isolated angiogenin polypeptide having at least one mutation to increase one or more ANG activities in the subject is provided. In certain aspects, the one or more ANG activities are selected from one or more of angiogenesis, ribonucleolytic activity, binding ANG receptor, activating tissue plasminogen activator, enhancing motor muscular function, enhancing neurite outgrowth, enhancing neurogenesis, enhancing survival of motor neurons, crossing the blood brain barrier, crossing the blood spinal cord barrier, enhancing survival of a subject having ALS and any combination thereof. In certain aspects, the isolated angiogenin polypeptide having at least one mutation has a D116H substitution or a Q117G substitution.

In certain exemplary embodiments, the isolated angiogenin polypeptide has at least two mutations to increase both ribonucleolytic activity and nuclear localization. In certain aspects, the isolated angiogenin polypeptide includes a D116H substitution and a modified nuclear localization sequence including for example, ³⁰KRRRG^{34 (M}30K), ³⁰RRRRG³⁴ (M30R), ³⁰MRRRK³⁴ (G34K), 30MRRRR³⁴ (G34R), ³⁰KRRRR³⁴ (M30KG34R), ³⁰RRRRK³⁴ (M30RG34K), ³⁰RRRRR³⁴ (M30RG34R) and ³⁰KRRRK³⁴ (M30KG34K). In certain aspects, the isolated angiogenin polypeptide includes a Q117G substitution and a modified nuclear localization sequence including for example, ³⁰KRRRG³⁴ (M30K), ³⁰RRRRG³⁴ (M30R), ³⁰MRRRK³⁴ (G34K), ³⁰MRRRR³⁴ (G34R), ³⁰KRRRG³⁴ (M30KG34R), ³⁰RRRRK³⁴ (M30RG34K), ³⁰RRRRR³⁴ (M30RG34R) and ³⁰KRRRK³⁴ (M30KG34K). In certain aspects, the subject lacks endogenous ANG or has one or more ANG mutations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts the conceptual framework of the interaction between ANG and its target cells. ANG, shown in yellow, can bind to both the receptor and the binding protein, shown in white and orange, respectively. The majority of the ANG and its binding protein complex will dissociate from the cell surface and activate tissue plasminogen activator (tPA) to produce plasmin, and induce cell invasion into the extracellular matrix. Binding to the 170 kDa receptor induces second messengers and triggers signal transduction. Upon binding, ANG is also internalized and translocated to the nucleus where it accumulates in the nucleolus. Each of these individual steps are necessary for angiogenesis.

FIG. 2 schematically depicts that ANG-stimulated rRNA transcription is a permissive factor for cell proliferation induced by other angiogenic factors. Without intending to be bound by scientific theory, the inventors' propose that ANG is a permissive factor for other angiogenic proteins to induce cell proliferation. Growth factors such as VEGF activate PI3K-AKT-mTOR pathway to enhance ribosomal protein production, but it is unclear how rRNA is proportionally increased. The experimentation described herein shows that ANG is translocated to the nucleus where it enhances rRNA transcription so that ribosome biogenesis can occur. Since ribosomes are essential for protein translation and cell proliferation, it has been demonstrated by the data presented herein that inhibiting ANG abolishes cell proliferation stimulated by other angiogenic factors including, but not limited to, bFGF and VEGF.

FIGS. 3A-3E depict the detection of ANG protein in the ventral horn area of spinal cord of ALS patients. Spinal cord sections of non-ALS subjects were from anonymous autopsy materials of Brigham and Women's Hospital. ALS-1 and -2 were from the National Disease Research Interchange. ALS-3 to −6 were from Dr. Bob Brown. ANG protein was stained by IHC with 26-2F (10 μg/ml at 4° C. for 16 h). (A) Images taken at 200× using Olympus camera DP70 attached to Olympus DC81 microscope. (B)-(E) Quantitative analyses by ImageJ software. Pixel-counting algorithm was used to obtain the chromogen specific areas that was then converted to μm². To obtain photon counts, the images were transformed in gray scale and the cumulative grayscale values of each of the three primary colors (red, green, and blue) was determined. (B) average motor neuron size. (C) total area covered by ANG-positive motor neuron in a ventral horn. (D) average photon counts in each motor neuron. E, total photon counts in an entire ventral horn.

FIGS. 4A-4F depict immunofluorescence (IF) detection of ANG protein in the ventral horn of human spinal cord. Human spinal cords from non-ALS controls and ALS patients were stained with anti-ANG mAb and anti-vWF pAb, respectively. Alexa 488-labeled goat anti-mouse IgG (green) and Alexa 555-labeled goat anti-rabbit IgG (red) were used to visualize ANG and vWF, respectively. Motor neurons and blood vessels were indicated by arrows and arrow heads, respectively.

FIGS. 5A-5B depict in situ hybridization (ISH) detection of ANG mRNA in spinal cord. Human spinal cords from non-ALS and ALS patients were stained with human ANG-specific riboprobe labeled with digoxiginin. An alkaline phosphatase-conjugated anti-digoxiginin antibody was used to visualize the signal. (A) images. (B) total photon counts of ANG mRNA in the ventral horn motor neurons determined with ImageJ.

FIG. 6A-6J depict the level of ANG protein and mRNA in mouse spinal cords. Spinal cords from wild-type (WT) and SOD1^{G93A} mice were stained with an anti-R165 and detected by IHC (A, B) or by IF (C, D). (E) quantitative analysis of IHC images from A and B. (F) Western blotting analysis of mouse ANG protein from spinal cord extracts. (G)-(J) A mouse ANG1-specific riboprobe was labeled with digoxiginin and used to detect the mRNA level of mouse ANG1. (K) Quantitative analysis of ISH images from (G) and (I).

FIG. 7A-7F depict decreased blood vessel size in the ALS spinal cord. Non-ALS and ALS human spinal cords (A)-(C), and WT and SOD1^{G93A} mice (D)-(F) were stained with an anti-vWF antibody. Blood vessels (brown staining) in a total of 10 microscopic areas of the ventral horns were counted (B) and (E), and the diameter of each counted vessels was measured (C) and (F), and the mean±SD values were shown.

FIG. 8 depicts that i.p. injected ANG reaches the CNS. PBS or human ANG protein, 10 μg/mouse, was injected i.p. into 11-week-old WT mice and SOD1^{G93A} mice. The animals were sacrificed 2.5 h post-injection after cardiac perfusion with PBS. The spinal cords were processed for IHC detection of human ANG with the mAb 26-2F.

FIGS. 9A-9D depict the effect of i.p. injection of ANG protein on SOD1^{G93A} mice. (A) qPCR detection of SOD1^{G93A} DNA. Data shown are the relative value to mouse GAPDH DNA. (B) ANG treatment enhances the muscle strength of the hind legs. (C) ANG treatment enhances motor function. Starting from 11 weeks of age, mice were treated with a weekly i.p. injection of WT or P112L ANG protein at 10 μg per mouse. PBS- (green), ANG- (red) and P112L mutant ANG- (black) treated mice were tested on a rotarod (20 rpm). (D) Survival curve of the three groups of mice.

FIGS. 10A-10B graphically depict the efficacy of three routes of administration. Wild-type ANG was administered into the SOD^{G93A} mice by subcutaneous (s.c.), i.p., or intravenous (i.v.) injection starting at week 10 and continued weekly. Rota-rod test was performed at week 11 and weekly afterward. Each group had six mice.

FIG. 11 graphically depicts the efficacy of three doses of ANG. Wild-type ANG was administered into the SOD^{G93A} mice by i.p. injection starting at week 10 at a dose of 0.1, 1, and 10 μg/mouse and continued weekly. Rotarod tests were performed at week 11 and weekly thereafter. Each group had five mice.

FIG. 12A-12D depicts the effect of ANG on motor neuron and blood vessels of SOD1^{G934} mice. WT and PBS or ANG-treated SOD1^{G93A} mice were sacrificed at week 15, at which time the ANG-treated mouse was at its peak performance on rotarod test. The lumbar region of the spinal cord was removed, fixed, embedded, and sectioned. (A) IHC with a human SOD-specific antibody (top panels); Nissl staining showing the motor neurons in the ventral horn (middle panels); and IHC with an anti-vWF antibody to show blood vessels (bottom panels). (B) Number of large motor neuron (>250 μm²) per section. (C) blood vessel density. (D) blood vessel size. A total of 500 vessels were measured in each sample. P values were calculated from Student t-test.

FIG. 13A-13D depict that ANG stimulates proliferation and differentiation of P19 cells. (A) IF detection of endogenous mouse ANG by R165. (B) human ANG was incubated with P19 cells for 2 hours and detected by IF with 26-2F. C, P19 cells were treated with or without ANG (50 ng/ml). On day 4, newly formed embryoid bodies were collected and counted. (D) P19 embryoid bodies were resuspended and cultured in the absence or presence of 50 ng/ml ANG for another 10 days. Neuronal processes are indicated by arrows.

FIG. 14A-14C depict the effect of angiogenin on NSC-34 motor neuron-like cells. (A) Nuclear translocation of exogenous angiogenin in NSC-34 cells. The cells were incubated with 0.5 μg/ml WT or P112L mutant human angiogenin for 2 hours, fixed and stained with mAb 26-2F and Alexa488-labeled 2^{nd} antibody and with DAPI. (B) Angiogenin stimulated NSC-34 cell proliferation. Cells were seeded in 48-well plates at 5×10³ cells per well and stimulated with angiogenin at 0.5, 1, and 10 μg/ml for 7 days. Cell numbers were determined by a Coulter counter. (C) Angiogenin stimulated neurite outgrowth. NSC-34 cells were cultured in the absence or presence of 0.5 μg/ml angiogenin for 7 days, and stained with 5,6-carboxyfluorescein diacetate.

FIG. 15A-15D depict the effect of ANG on P19 cell apoptosis. Cells were serum-starved in the absence or presence of ANG for 24 hours. Apoptotic cells were stained by EB (A), counted and the percentage calculated (B). (C) Caspase activities were measured using Caspase-Glo 3/7 Assay kit (Promega). (D) P19 cells were cultured on PA6 cells in the presence of 0.5 μM retinoic acid for 10 days. The cells were then put at 20° C. for 40 minutes with or without ANG (0.5 μg/ml), and subject to IF detection of neurofilaments.

FIG. 16 depicts the detection of ANG in the neuromuscular junction (NMJ). Mouse tibialis anterior muscle was removed and frozen section of 40 μm cut. ANG was stained with pAb R165 and Alexa 488-goat anti rabbit IgG. NF were stained with anti-NF medium subunit mAb and Alex a 555-goat anti mouse IgG.

FIG. 17 depicts the amino acid sequences of mammalian ANGs. By convention, the first amino acid in the mature protein is designated as 1. The signal peptide is underlined. Mutations are bracketed in red. Human is set forth as SEQ ID NO:1; chimpanzee is set forth as SEQ ID NO:2; gorilla is set forth as SEQ ID NO:3; mouse is set forth as SEQ ID NO:4; rat is set forth as SEQ ID NO:5; bovine is set forth as SEQ ID NO:6; porcine is set forth as SEQ ID NO:7.

FIG. 18A is and SDS-PAGE and Coomassie blue staining of WT, M30K, G34K and M30G34K angiogenin variants. FIG. 18B is a graph showing the ribonucleolytic activity of WT and angiogenin variants towards yeast tRNA.

FIG. 19 shows pictures of human umbilical vein endothelial cells cultured on Matrigel with WT ANG and angiogenin variants.

FIG. 20A shows P19 cells incubated with WT and G34K ANG for different times and detected by IF with mab 26-2F. FIG. 20B is a graph showing P19 cell numbers after incubation with WT and G34K ANG for 4 days.

FIG. 21 is a graph P19 cell numbers treated with tunicamycin in the presence of WT ANG and G34K ANG.

FIG. 22 is a graph showing time on rotarod for SOD1G^{93A} mice injected with WT ANG and G34K ANG.

FIG. 23 is a graph showing weight loss prevention of SOD1G⁹³⁴ mice.

FIG. 24 is a Kaplan-Meyer plot showing probability of survival of SOD1G^{93A} mice.

### DETAILED DESCRIPTION

In certain exemplary embodiments, methods and compositions including one or more ANG polypeptides or angiogenin variants or modifications thereof or fragments thereof and/or one or more nucleic acid sequences encoding one or more ANG polypeptides or angiogenin variants or modifications thereof or fragments thereof are provided for treating a neurological disorder (e.g., ALS) in a subject. As used herein, the term “ANG polypeptide” refers to an angiogenic ribonuclease or variants or modifications (e.g., ANG in humans) or portion or fragment thereof having one or more ANG properties: 1) promoting angiogenesis; 2) having ribonucleolytic activity; 3) binding the ANG receptor; 4) activating tissue plasminogen activator; 5) enhancing motor muscular function (e.g., in an individual having ALS); 6) enhancing neurite outgrowth (e.g., in an individual having ALS); 7) enhancing neurogenesis (e.g., in an individual having ALS); 8) enhancing survival of motor neurons (e.g., in an individual having ALS); 9) crossing the BBB; 10) crossing the BSCB; 11) enhancing survival of in an individual having ALS; and/or 11) enhancing and/or restoring one or more functions described above in an individual (e.g., an individual having an ANG mutation or deletion). In certain exemplary embodiments, an ANG polypeptide can eliminate, ameliorate and/or decrease one or more symptoms associated with ALS: 1) degeneration of motor neurons; 2) muscle weakness; 3) muscle atrophy; 4) motor neuron degeneration (e.g., upper- and/or lower motor neurons); 5) fasciculation development; 6) frontotemporal dementia and/or 7) decreased lifespan. “Fragments” refer to a portion of angiogenin protein whether modified or a variant thereof and include a contiguous stretch of amino acid residues of at least 5 amino acids, 10 amino acids, 20 amino acids, 30 amino acids, 40 amino acids and any number in between. “Variants” refer to proteins having amino acid sequences similar, i.e. homologous to the wild type angiogenin, typically wild type human angiogenin. Homologies can include at least about 70%, 80%, 90%, 95%, 96%, 97%, 98%, 99% and greater. Variants are typically altered in one or more amino acid sequences by insertion, addition, deletion or substitution techniques well known to those skilled in the art. Preferably, the fragments and variants exhibit one nor more properties of an angiogenin polypeptide described above.

As used herein, the terms “subject,” “individual” and “host” are intended to include living organisms such as mammals. Examples of subjects and hosts include, but are not limited to, horses, cows, sheep, pigs, goats, dogs, cats, rabbits, guinea pigs, rats, mice, gerbils, non-human primates (e.g., macaques), humans and the like, non-mammals, including, e.g., non-mammalian vertebrates, such as birds (e.g., chickens or ducks) fish or frogs (e.g., Xenopus), and non-mammalian invertebrates, as well as transgenic species thereof.

As used herein, the terms “neurological disorder” and “neurological disease” include, but are not limited to, neuromuscular disorders, Alzheimer's disease, aphasia, Bell's palsy, Creutzfeldt-Jacob disease, cerebrovascular disease, encephalitis, epilepsy, prion disease, Huntington's disease, pain, phobia, movement disorders (e.g., Parkinson's disease), sleep disorders, Tourette syndrome, multiple sclerosis, neural tumors, neural autoimmune disorders (e.g., multiple sclerosis) pediatric neural disorders (e.g., autism, dyslexia, cerebral palsy and the like) and the like. As used herein, the terms “neuromuscular disorder” and “motor neuron disorder” include, but are not limited to, disorders such as ALS, Guillain-Barre syndrome, Charcot-Marie-Tooth disease, spinal muscular atrophy (SMA), muscular dystrophy, spastic paraplegia and the like.

In certain exemplary embodiments, a therapeutic amount of one or more ANG polypeptides and/or angiogenin variants or modifications thereof or fragments thereof and/or one or more nucleic acid sequences encoding one or more ANG polypeptides and/or angiogenin variants or modifications thereof or fragments thereof is administered to an individual in need thereof, e.g., for the treatment of a neurological disorder such as, e.g., ALS. The one or more ANG polypeptides and/or angiogenin variants or modifications thereof or fragments thereof and/or one or more nucleic acid sequences encoding one or more ANG polypeptide and/or angiogenin variants or modifications thereof or fragments thereof described herein can be incorporated into pharmaceutical compositions suitable for administration. Such compositions typically comprise the one or more nucleic acid molecules or polypeptides and a pharmaceutically acceptable carrier.

According to certain exemplary embodiments, the ANG polypeptides and/or angiogenin variants or modifications thereof or fragments thereof include ANG polypeptides and/or angiogenin variants or modifications thereof or fragments thereof that have been chemically modified to include or attach polyethylene glycol (PEG) to the ANG polypeptides and/or angiogenin variants or modifications thereof or fragments thereof in a process referred to as PEGylation. Specific advantages of PEGylation include increased efficacy, reduced dosing frequency, reduced toxicity, reduced immunogenicity, reduced side effects, increased stability, increased shelf-life, increased half-life and enhanced solubility. The ANG polypeptides and/or angiogenin variants or modifications thereof or fragments thereof may be PEGylated directly or through a linker according to the methods known to those of skill in the art such as Davis, Adv. Drug Deliv. Rev. 54, 457-458 (2002), Veronese, Bioorg. Med. Chem. Lett, 12, 177-180 (2002), Harris, Adv. Drug. Deliv. Rev. 54, 459-476 (2002), Chapman, Nature Biotechnology 17, 780-783 (1999), and Sato, Adv. Drug Deliv. Rev. 54, 487-504 (2002) hereby incorporated by reference in their entireties and other references readily available to those of skill in the art. Similarly, the ANG polypeptides and/or angiogenin variants or modifications thereof or fragments thereof can be chemically glysocylated insofar as saccharides are linked to the ANG polypeptides and/or angiogenin variants or modifications thereof or fragments thereof using methods known to those of skill in the art. Examples of glycosylation include N-linked glycosylation and O-linked glycosylation. Specific advantages of glysocylation include increased efficacy, reduced dosing frequency, reduced toxicity, reduced immunogenicity, reduced side effects, increased stability, increased shelf-life, increased half-life and enhanced solubility. Further embodiments of the ANG polypeptides and/or angiogenin variants or modifications thereof or fragments thereof include dimers, trimers, oligomers, etc. thereof. It is to be understood that modifications of the ANG polypeptides and/or angiogenin variants or modifications thereof or fragments thereof include modifications, chemical, physical or otherwise, to a core angiogenin molecule or variant or modification or fragment thereof used by those of skill in the art to increase efficacy, reduce dosing frequency, reduced toxicity, reduced immunogenicity, reduced side effects, increased stability, increased shelf-life, increased half-life and enhanced solubility such as PEGylation or glycosylation or dimerization other methods known to those of skill in the art.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutically acceptable carriers and their formulations are known to those skilled in the art and described, for example, in Remington's Pharmaceutical Sciences, (19th edition), ed. A. Gennaro, 1995, Mack Publishing Company, Easton, Pa.

In certain exemplary embodiments, pharmaceutical formulations of a therapeutically effective amount of one or more ANG polypeptides and/or angiogenin variants or modifications thereof or fragments thereof or one or more nucleic acid sequences encoding one or more ANG polypeptides and/or angiogenin variants or modifications or fragments thereof, or pharmaceutically acceptable salts thereof, are administered by intravenous injection, intraperitoneal injection, oral administration or by other parenteral routes (e.g. intradermal, subcutaneous, oral (e.g., inhalation), transdermal (topical), transmucosal, and rectal administration), or by intrathecal and intraventricular injections into the CNS, in an admixture with a pharmaceutically acceptable carrier adapted for the route of administration.

In certain exemplary embodiments, pharmaceutical formulations of a therapeutically effective amount of one or more ANG polypeptides and/or angiogenin variants or modifications or fragments thereof or one or more nucleic acid sequences encoding one or more ANG polypeptides and/or angiogenin variants or modifications or fragments thereof, or pharmaceutically acceptable salts thereof, are administered directly to the central nervous system (CNS), e.g., intrathecally, intracerebrally, via the olfactory nerve, via the olfactory epithelium, via the spinal cord and the like. Direct administration to the CNS can be performed using an implantable infusion pump or a transplanted delivery scaffold, for example. Implantable infusion pumps are commercially available (Medtronic, Inc., Minneapolis, Minn.) and are described in U.S. Pat. Nos. 5,711,316, 5,814,014 and 7,232,435. Delivery scaffolds for use in the CNS are known in the art and described in, e.g., Lee et al. (2006) Toxicol. Appl. Pharm. 215:64; Proceedings of the IEEE 31^{st} Annual Northeast Bioengineering Conference (2005) 1-3, ISBN: 0-7803-9105-5 INSPEC Accession Number: 8487652, DOI:10.1109/NEBC.2005.1431898, Posted online: 2005-05-23 09:06:59.0; DeLaporte and Shea (2007) Adv. Drug Deliv. Rev. 59:292; and Nomura et al. (2006). J. Neurotrauma 23:496.

Solutions or suspensions used for parenteral, intradermal, subcutaneous or central nervous system application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Methods well known in the art for making formulations are found, for example, in Remington's Pharmaceutical Sciences (19th edition), ed. A. Gennaro, 1995, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain exemplary embodiments, isotonic agents, for example, sugars, polyalcohols such as mannitol, sorbitol, and/or sodium chloride, will be included in the composition. Prolonged absorption of the injectable compositions can be brought about by including in the composition an agent which delays absorption, for example, aluminum monostearate and gelatin.

Sterile injectable solutions can be prepared by incorporating one or more ANG polypeptides and/or angiogenin variants or one or more nucleic acid sequences encoding one or more ANG polypeptides and/or angiogenin variants described herein in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: A binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, the one or more ANG polypeptides and/or angiogenin variants or modifications or fragments thereof one or more nucleic acid sequences encoding one or more ANG polypeptides and/or angiogenin variants or modifications or fragments thereof described herein are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The one or more ANG polypeptides and/or angiogenin variants or modifications or fragments thereof or one or more nucleic acid sequences encoding one or more ANG polypeptides and/or angiogenin variants or modifications or fragments thereof described herein can also be prepared in the form of suppositories (e.g., with conventional suppository bases such as cocoa butter and other glycerides) or retention enemas for rectal delivery.

In one embodiment, the one or more ANG polypeptides and/or angiogenin variants or modifications or fragments thereof or one or more nucleic acid sequences encoding one or more ANG polypeptides and/or angiogenin variants or modifications or fragments thereof described herein are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral, parenteral or CNS direct delivery compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of one or more ANG polypeptides and/or angiogenin variants or modifications or fragments thereof and/or nucleic acid sequences encoding one or more ANG polypeptides and/or angiogenin variants or modifications or fragments thereof described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In certain exemplary embodiments, a method for treatment of a neurological disorder (e.g., ALS), includes the step of administering a therapeutically effective amount of an agent (e.g., one or more ANG polypeptides and/or angiogenin variants or modifications or fragments thereof or one or more nucleic acid sequences encoding one or more ANG polypeptides and/or angiogenin variants or modifications or fragments thereof) to a subject. As defined herein, a therapeutically effective amount of agent (i.e., an effective dosage) ranges from about 0.0001 to 30 mg/kg body weight, from about 0.001 to 25 mg/kg body weight, from about 0.01 to 20 mg/kg body weight, from about 0.1 to 15 mg/kg body weight, or from about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of one or more ANG polypeptides and/or angiogenin variants or modifications or fragments thereof or one or more nucleic acid sequences encoding one or more ANG polypeptides and/or angiogenin variants or modificatyions or fragments thereof can include a single treatment or, in certain exemplary embodiments, can include a series of treatments. It will also be appreciated that the effective dosage of agent used for treatment may increase or decrease over the course of a particular treatment. Changes in dosage may result from the results of diagnostic assays as described herein. The pharmaceutical compositions can be included in a container, pack, or dispenser together with instructions for administration.

In certain exemplary embodiments, vectors such as, for example, expression vectors, containing a nucleic acid encoding one or more ANG polypeptides and/or angiogenin variants or modifications or fragments thereof described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more ANG polypeptides and/or angiogenin variants or modifications or fragments thereof described herein) in a form suitable for expression of the nucleic acid sequence in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence encoding one or more ANG polypeptides and/or angiogenin variants or modifications or fragments thereof is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors described herein can be introduced into host cells to thereby produce proteins or portions thereof, including fusion proteins or portions thereof, encoded by nucleic acids as described herein (e.g., one or more ANG polypeptides and/or angiogenin variants).

In certain exemplary embodiments, nucleic acid molecules described herein can be inserted into vectors and used as gene therapy vectors. Gene therapy vectors can be delivered to a subject by, for example, intravenous injection, local administration (see, e.g., U.S. Pat. No. 5,328,470), or by stereotactic injection (see, e.g., Chen et al. (1994) Proc. Natl. Acad. Sci. U.S.A. 91:3054). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, adeno-associated virus vectors, and the like, the pharmaceutical preparation can include one or more cells which produce the gene delivery system (See Gardlik et al. (2005) Med. Sci. Mon. 11:110; Salmons and Gunsberg (1993) Hu. Gene Ther. 4:129; and Wang et al. (2005) J. Virol. 79:10999 for reviews of gene therapy vectors).

### Herpesvirus Vectors

Herpesvirus is trophic for cells of the nervous system (e.g., neural cells). Various defective (i.e., non-replicating, non-infectious) herpesvirus vectors have been described, such as a defective herpes virus 1 (HSV-1) vector (WO 94/21807, WO 92/05263).

### Adenovirus Vectors

Adenoviruses are eukaryotic DNA viruses that can be modified to efficiently deliver a nucleic acid of the invention to a variety of cell types in vivo, and have been used extensively in gene therapy protocols, including for targeting genes to neural cells. Various serotypes of adenovirus exist. Of these serotypes, preference is given to using type 2 or type 5 human adenoviruses (Ad-2 or Ad-5) or adenoviruses of animal origin (see WO 94/26914). Adenoviruses of animal origin include, but are not limited to, adenoviruses of canine, bovine, murine (e.g., May 1), ovine, porcine, avian, and simian (e.g., SAV) origin. In certain exemplary embodiments, the adenovirus of animal origin is a canine adenovirus, such as a CAV2 adenovirus (e.g., Manhattan or A26/61 strain (ATCC VR-800)). Various replication defective adenovirus and minimum adenovirus vectors have been described for gene therapy (See, e.g., WO 94/26914, WO 95/02697, WO 94/28938, WO 94/28152, WO 94/12649, WO 95/02697 WO 96/22378).

### Adeno-Associated Viruses

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation. The use of vectors derived from AAVs for transferring genes in vitro and in vivo has been described (See, e.g., WO 91/18088; WO 93/09239; U.S. Pat. No. 4,797,368, U.S. Pat. No. 5,139,941, EP 488528). Replication defective recombinant AAVs can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced can then be purified by standard techniques.

### Retrovirus Vectors

In certain exemplary embodiments, a gene expressing ANG or a portion or mutant or variant form thereof can be introduced in a retroviral vector (See, e.g., U.S. Pat. No. 5,399,346; U.S. Pat. No. 4,650,764; U.S. Pat. No. 4,980,289; U.S. Pat. No. 5,124,263; EP 453242, EP178220; WO 95/07358). Retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, for example, murine Moloney leukemia virus, murine Moloney sarcoma virus, Harvey sarcoma virus, spleen necrosis virus, Rous sarcoma virus, Friend virus and the like. Suitable packaging cell lines have been described, such as, for example, the cell line PA317 (U.S. Pat. No. 4,861,719); the PsiCRIP cell line (WO 90/02806) and the GP+envAm-12 cell line (WO 89/07150). In addition, the recombinant retroviral vectors can contain modifications within the LTRs for suppressing transcriptional activity as well as extensive encapsidation sequences which may include a part of the gag gene. Recombinant retroviral vectors are purified by standard techniques known to those having ordinary skill in the art. Retrovirus vectors can also be introduced by recombinant DNA viruses, which permits one cycle of retroviral replication and amplifies transfection efficiency (See, e.g., WO 95/22617, WO 95/26411, WO 96/39036, WO 97/19182).

In certain exemplary embodiments, lentiviral vectors can be used to provide highly effective expression of a gene of interest as lentiviruses can change the expression of their target cell's gene for up to six months. They can be used, for example, in non-dividing or terminally differentiated cells such as neurons, macrophages, hematopoietic stem cells, retinal photoreceptors and muscle and liver cells, cell types for which previous gene therapy methods could not be used. The vectors can efficiently transduce dividing and non-dividing cells in these tissues, and maintain long-term expression of the gene of interest. Lentiviral packaging cell lines are available and known generally in the art. They facilitate the production of high-titer lentivirus vectors for gene therapy. An example is a tetracycline-inducible VSV-G pseudotyped lentivirus packaging cell line which can generate virus particles at titers greater than 10⁶ IU/ml for at least 3 to 4 days. The vector produced by the inducible cell line can be concentrated as needed for efficiently transducing non-dividing cells in vitro and in vivo.

### Non-Viral Vectors

A vector can be introduced in vivo in a non-viral vector, e.g., by lipofection, with other transfection facilitating agents (peptides, polymers and the like), or as naked DNA. Synthetic cationic lipids can be used to prepare liposomes for in vivo transfection, with targeting in some instances (Feigner, et. al., 1987; Feigner and Ringold, 1989; see Mackey, et al., 1988; Ulmer et al., 1993). Useful lipid compounds and compositions for transfer of nucleic acids are described in WO 95/18863, WO 96/17823 and in U.S. Pat. No. 5,459,127. Other molecules are also useful for facilitating transfection of a nucleic acid in vivo, such as, e.g., a cationic oligopeptide (See, e.g., WO 95/21931), peptides derived from DNA binding proteins (See, e.g., WO 96/25508) a cationic polymer (See, e.g., WO 95/21931) and the like. A relatively low voltage, high efficiency in vivo DNA transfer technique, termed electrotransfer, has been described (See, e.g., WO 99/01157; WO 99/01158; WO 99/01175). DNA vectors for gene therapy can be introduced into the desired host cells by methods known in the art, e.g., electroporation, microinjection, cell fusion, DEAE dextran, calcium phosphate precipitation, use of a gene gun (ballistic transfection), or use of a DNA vector transporter (See, e.g., Canadian Patent Application No. 2,012,311). Receptor-mediated DNA delivery approaches can also be used. U.S. Pat. Nos. 5,580,859 and 5,589,466 disclose delivery of exogenous DNA sequences, free of transfection facilitating agents, in a mammal.

Expression vectors described herein can be designed for expression of one or more ANG polypeptides in prokaryotic or eukaryotic cells. For example, one or more vectors encoding one or more ANG polypeptides can be expressed in bacterial cells such as E. coli, insect cells (e.g., using baculovirus expression vectors), yeast cells or mammalian cells. Suitable host cells are discussed further in Goeddel, Gene Expression Technology Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40); pMAL (New England Biolabs, Beverly, Mass.); and pRIT5 (Pharmacia, Piscataway, N.J.) which fuse glutathione S-transferase (GST), maltose E binding protein, or protein A, respectively, to the target recombinant protein.

In certain exemplary embodiments, the expression vector encoding one or more ANG polypeptides and/or angiogenin variants is a yeast expression vector. Examples of vectors for expression in yeast S. cerevisiae include pYepSec1 (Baldari, et. al., (1987) EMBO J. 6:229-234); pMFa (Kurjan and Herskowitz, (1982) Cell 30:933-943); pJRY88 (Schultz et al., (1987) Gene 54:113-123); pYES2 (Invitrogen Corporation, San Diego, Calif.); and picZ (Invitrogen Corporation).

Alternatively, one or more ANG polypeptides and/or angiogenin variants or

modifications or fragments thereof can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In certain exemplary embodiments, a nucleic acid described herein is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, adenovirus 2, cytomegalovirus and simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In certain exemplary embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include lymphoid-specific promoters (Calamc and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374) and the α-fetoprotein promoter (Campes and Tilghman (1989) Genes Dev. 3:537).

In certain exemplary embodiments, host cells into which a recombinant expression vector of the invention has been introduced are provided. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, one or more ANG polypeptides and/or angiogenin variants or modifications or fragments thereof can be expressed in bacterial cells such as E. coli, viral cells such as retroviral cells, insect cells, yeast or mammalian cells (such as Chinese hamster ovary cells (CHO) or COS cells). Other suitable host cells are known to those skilled in the art.

Delivery of nucleic acids described herein (e.g., vector DNA) can be by any suitable method in the art. For example, delivery may be by injection, gene gun, by application of the nucleic acid in a gel, oil, or cream, by electroporation, using lipid-based transfection reagents, or by any other suitable transfection method.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEIT™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

Embodiments of the invention are directed to a first nucleic acid (e.g., a nucleic acid sequence encoding one or more ANG polypeptides and/or angiogenin variants or modifications or fragments thereof) or polypeptide sequence (e.g., one or more ANG polypeptides and/or angiogenin variants or modifications or fragments thereof) having a certain sequence identity or percent homology to a second nucleic acid or polypeptide sequence, respectively.

Techniques for determining nucleic acid and amino acid “sequence identity” are known in the art. Typically, such techniques include determining the nucleotide sequence of genomic DNA, mRNA or cDNA made from an mRNA for a gene and/or determining the amino acid sequence that it encodes, and comparing one or both of these sequences to a second nucleotide or amino acid sequence, as appropriate. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986) Nucl. Acids Res. 14:6745. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.).

One method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the NCBI/NLM web site.

Alternatively, homology can be determined by hybridization of polynucleotides under conditions that form stable duplexes between homologous regions, followed by digestion with single-stranded-specific nuclease(s), and size determination of the digested fragments. Two DNA sequences, or two polypeptide sequences are “substantially homologous” to each other when the sequences exhibit at least about 80%-85%, at least about 85%-90%, at least about 90%-95%, or at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning: A Laboratory Manual, Second Edition, (1989) Cold Spring Harbor, N.Y.; Nucleic Acid Hybridization: A Practical Approach, editors B. D. Hames and S. J. Higgins, (1985) Oxford; Washington, D.C.; IRL Press.

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., supra). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization, supra).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as varying wash conditions. The selection of a particular set of hybridization conditions is selected following standard methods in the art (see, for example, Sambrook et al., supra).

As used herein, the term “hybridizes under stringent conditions” is intended to describe conditions for hybridization and washing under which nucleotide sequences at least 60% identical to each other typically remain hybridized to each other. In one aspect, the conditions are such that sequences at least about 70%, at least about 80%, at least about 85% or 90% or more identical to each other typically remain hybridized to each other. Such stringent conditions are known to those skilled in the art and can be found in Current Protocols in Molecular Biology, John Wiley & Sons, NY (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS at 50° C., at 55° C., or at 60° C. or 65° C.

In certain exemplary embodiments screening assays for identifying modulators, i.e., candidate or test compounds or agents (e.g., antibodies, peptides, cyclic peptides, peptidomimetics, small molecules, small organic molecules, or other drugs) which have a stimulatory effect on one or more ANG polypeptides and/or angiogenin variants and an inhibitory effect on one or more neurodegenerative diseases (e.g., ALS) are provided.

As used herein, the term “small molecule” refers to a molecule, either naturally occurring or synthetic, that has a molecular weight of more than about 25 daltons and less than about 3000 daltons, usually less than about 2500 daltons, more usually less than about 2000 daltons, usually between about 100 to about 1000 daltons, more usually between about 200 to about 500 daltons.

In certain exemplary embodiments, assays for screening candidate or test compounds which bind to or modulate (e.g., stimulate) one or more ANG polypeptides and/or angiogenin variants or modifications or fragments thereof and modulate (e.g., inhibit) one or more neurodegenerative diseases (e.g., ALS) are provided. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables, and accompanying claims.

### Example 1

### Angiogenin

ANG was originally isolated from the conditioned medium of HT-29 human colon adenocarcinoma cells based solely on its angiogenic activity in the chicken embryo chorioallantoic membrane (CAM) angiogenesis assay (Fett et al. (1985) Biochemistry 24:5480). Subsequently, ANG has been found to have a wide tissue distribution with the liver being the major source for circulating ANG (Weiner et al. (1987) Science 237:280). ANG is a member of the pancreatic ribonuclease A (RNase A) superfamily with a 33% amino acid identity and an overall homology of 56% to that of RNase A (Strydom et al. (1985) Biochemistry 24:5486). ANG has a unique ribonucleolytic activity that is several orders of magnitude lower than that of RNase A but is important for its biological activity (Shapiro et al. (1986) Biochemistry 25:3527). The amino acid residues important for catalysis are conserved in all vertebrate ANG from fish to human (Riordan (2001) Methods Enzymol. 341:263). Extensive work on site-directed mutagenesis has shown that ANG variants with reduced enzymatic activity invariably have reduced angiogenic activity (Shapiro et al. (1986) Biochemistry 25:3527; Curran et al. (1993) Biochim. Biophys. Acta. 1202:281; Hallahan et al. (1992) Biochemistry 31:8022; Hallahan et al. (1991) Proc. Natl. Acad. Sci. USA 88:2222; Harper et al. (1990) Biochemistry 29:7297; Shapiro et al. (1989) Biochemistry 28:1726; Shapiro and Vallee (1989) Biochemistry 28:7401; Shapiro and Vallee (1992) Biochemistry 31:12477; Shapiro et al. (1987) Proc. Natl. Acad. Sci. USA 84:8783). Structural work indicated one of the reasons for ANG to have a reduced ribonucleolytic activity is that the side chain of Gln 117 occupies part of the enzymatic active site so that substrate binding is compromised (Acharya et al. (1994) Proc. Natl. Acad. Sci. USA 91:2915; Russo et al. (1994) Proc. Natl. Acad. Sci. USA 91:2920).

ANG is angiogenic, whereas the prototype family member RNase A is not. Two important structural differences between ANG and RNase A are responsible for this discrepancy. The first is the segment from amino acid residues 59 to 68 that forms the receptor binding site (Hallahan et al. (1991) Proc. Natl. Acad. Sci. USA 88:2222; Hu et al. (1991) Proc. Natl. Acad. Sci. USA 88:2227) in ANG and is very different in RNase A (Acharya et al. (1994) Proc. Natl. Acad. Sci. USA 91:2915; Acharya et al. (1995) Proc. Natl. Acad. Sci. USA 92:2949). Therefore ANG binds to its target cells (including endothelial cells, cancer cells and motor neurons) but RNase A does not. ANG binds to endothelial cells specifically (Badet et al. (1989) Proc. Natl. Acad. Sci. USA 86:8427) and induces second messenger responses including diacylglycerol and prostacyclin (Bicknell and Vallee (1988) Proc. Natl. Acad. Sci. USA 85:5961; Bicknell and Vallee (1989) Proc. Natl. Acad. Sci. USA 86:1573), and activates MAP kinase (Liu et al. (2001) Biochem. Biophys. Res. Commun. 287:305) and AKT (Kim et al. (2007) Biochem. Biophys. Res. Commun. 352:509).

Another structural difference between ANG and RNase A is that ANG has a nuclear localization signal (NLS) consisting of 29IMRRRGL35 (SEQ ID NO:8), whereas RNase A does not (Moroianu and Riordan (1994) Biochem. Biophys. Res. Commun. 203:1765). Therefore ANG undergoes nuclear translocation in endothelial cells where it accumulates in the nucleolus (Moroianu and Riordan, J. F. (1994) Proc. Natl. Acad. Sci. USA 91:1677; Hu and Riordan (2000) J. Cell. Biochem. 76:452), binds to the promoter region of ribosomal DNA (rDNA) and stimulates ribosomal RNA (rRNA) transcription (Xu et al. (2002) Biochem. Biophys. Res. Commun. 294:287, Xu et al. (2003) Biochemistry 42:121), an essential step for ribosome biogenesis and therefore for protein translation and cell proliferation.

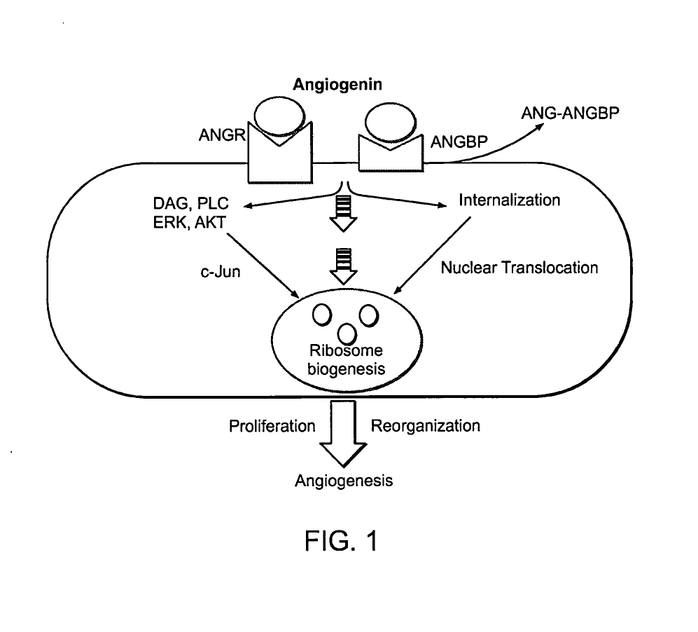

An ANG binding protein has been identified from the surface of endothelial cells (Hu et al. (1991) Proc. Natl. Acad. Sci. USA 88:2227) and has been characterized to be a type of smooth muscle actin (Hu et al. (1993) Proc. Natl. Acad. Sci. USA 90:1217; Moroianu et al. (1993) Proc. Natl. Acad. Sci. USA 90:3815). An approximately 170 kDa ANG receptor has also been identified from the endothelial cell surface to mediate nuclear translocation of ANG and cell proliferation (Hu et al. (1997) Proc. Natl. Acad. Sci. USA 94:2204). Expression of the binding protein and the receptor on endothelial cells seems to be mutually exclusive. The binding protein is expressed on the surface of confluent cells. Binding of ANG to the binding protein activates tissue plasminogen activator (tPA) (Hu and Riordan (1993) Biochem. Biophys. Res. Commun. 197:682) thereby inducing cell invasion and migration (Hu et al. (1994) Proc. Natl. Acad. Sci. USA 91:12096). After the leading cells migrate away, the local cell density decreases which triggers the expression of an ANG receptor. Binding of ANG to the receptor stimulates cell proliferation so that the gap created by the migrating cells is filled. Therefore, ANG is a multifunctional angiogenic molecule that plays a role in several steps in the angiogenesis process including cell invasion, proliferation, and tube formation. FIG. 1 summarizes the findings presented herein regarding the mechanism of ANG-induced angiogenesis.

### Example 2

### Role of ANG in rRNA Transcription

ANG has been shown to undergo nuclear translocation in endothelial cells (Moroianu and Riordan, J. F. (1994) Proc. Natl. Acad. Sci. USA 91:1677; Hu and Riordan (2000) J. Cell. Biochem. 76:452; Li et al. (1997) Biochem. Biophys. Res. Commun. 238:305) and in various types of human cancer cells (Tsuji et al. (2005) Cancer Res. 65:1352; Yoshioka et al. (2006) Proc. Natl. Acad. Sci. USA 103:14519). Nuclear translocation of ANG in endothelial cells is under tight regulation and is cell density-dependent. It decreases as cell density increases and ceases when cells are confluent (Hu and Riordan (2000) J. Cell. Biochem. 76:452; Hu et al. (1997) Proc. Natl. Acad. Sci. USA 94:2204). Nuclear translocation of ANG occurs through receptor-mediated endocytosis (Moroianu and Riordan, J. F. (1994) Proc. Natl. Acad. Sci. USA 91:1677) and is independent of microtubules systems and lysosomal processing (Li et al. (1997) Biochem. Biophys. Res. Commun. 238:305) and ANG seems to enter the nuclear pore by the classic nuclear pore input route (Moroianu and Riordan (1994) Biochem. Biophys. Res. Commun. 203:1765). Nuclear translocation of exogenous ANG was very fast. When exogenous ANG is added to the cell culture, nuclear ANG is detectable within 2 minutes and is saturated in 30 minutes (Hu and Riordan (2000) J. Cell. Biochem. 76:452). Upon arriving at the nucleus, ANG accumulates in the nucleolus (Moroianu and Riordan, J. F. (1994) Proc. Natl. Acad. Sci. USA 91:1677) where ribosome biogenesis takes place. Nuclear ANG has been shown to bind to the promoter region of rDNA (Xu et al. (2003) Biochemistry 42:121) and stimulates rRNA transcription (Xu et al. (2002) Biochem. Biophys. Res. Commun. 294:287; Kishimoto et al. (2005) Oncogene 24:445).

Cell growth requires the production of new ribosomes. Ribosomal biogenesis is a process involving rRNA transcription, processing of the pre-rRNA precursor and assembly of the mature rRNA with ribosomal proteins (Comai (1999) Braz. J. Med. Biol. Res. 32:1473; Melese and Xue (1995) Curr. Opin. Cell. Biol. 7:319; Stoykova et al. (1985) J. Neurochem. 45:1667). The rate-limiting step in ribosome biogenesis is the synthesis of rRNA. Therefore, rRNA transcription is an important aspect of growth control. When cells are quiescent, the overall rate of protein accumulation is reduced. On mitogenic stimulation the synthesis of rRNA is accelerated, and the production of ribosomal proteins and translation factors increases before cells reach S phase (Rosenwald (1996) Cancer Letters 102:113; Clarke et al. (1996) J. Biol. Chem. 271:22189; Rosenwald (1996) Bioessays 18:243). The rate of growth is directly proportional to the rate of protein accumulation and this is related to ribosome content (Baxter and Stanners (1978) J. Cell Physiol. 96:139). As ribosome biogenesis is a limiting factor for cell duplication, the rate of cell proliferation could be controlled by modulating the expression of nucleolar proteins involved in rDNA gene transcription, rRNA processing, and transport of transcripts to the cytoplasm. Nuclear localization of those nucleolar proteins from the cytoplasm or from outside of the cells would then be an important factor to control ribosome biogenesis.

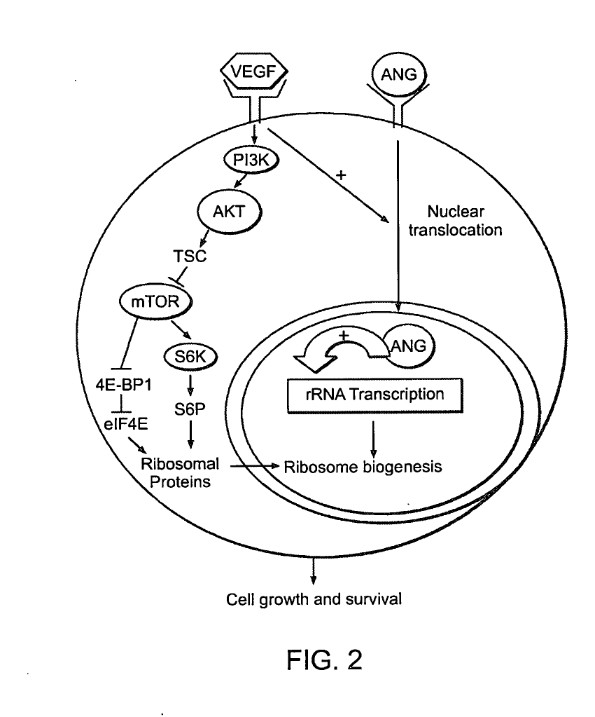

ANG appears to be one of the proteins that is translocated to the nucleus where it regulates rRNA transcription in its targeting cells. Therefore, ANG-stimulated rRNA transcription has been proposed as a general requirement for angiogenesis and is a common crossroad that all the angiogenic factors need to go through (Kishimoto et al. (2005) Oncogene 24:445). In other words, ANG is a permissive factor for other angiogenic factors. Experimental evidence for this contention includes: (I) nuclear translocation of endogenous ANG in endothelial cells is stimulated by other angiogenic factors including acidic fibroblast growth factor (aFGF), basic fibroblast growth factor (bFGF), vascular endothelial growth factor (VEGF) and epidermal growth factor (EGF) (Kishimoto et al. (2005) Oncogene 24:445); (2) knocking-down ANG expression in endothelial cells inhibits bFGF- and VEGF-induced cell proliferation accompanied with a decrease in rRNA transcription. Addition of exogenous ANG can completely restore the proliferative activity of these angiogenic factors (Kishimoto et al. (2005) Oncogene 24:445); (3) ANG-specific inhibitors had no effect on binding of VEGF and bFGF to their receptors but inhibit angiogenesis induced by them (Hirukawa et al. (2005) Clin. Cancer Res. 11:8745). FIG. 2 summarizes the function of ANG-stimulated rRNA transcription in cell proliferation.

### Example 3

### ANG Mutation is ALS Patients

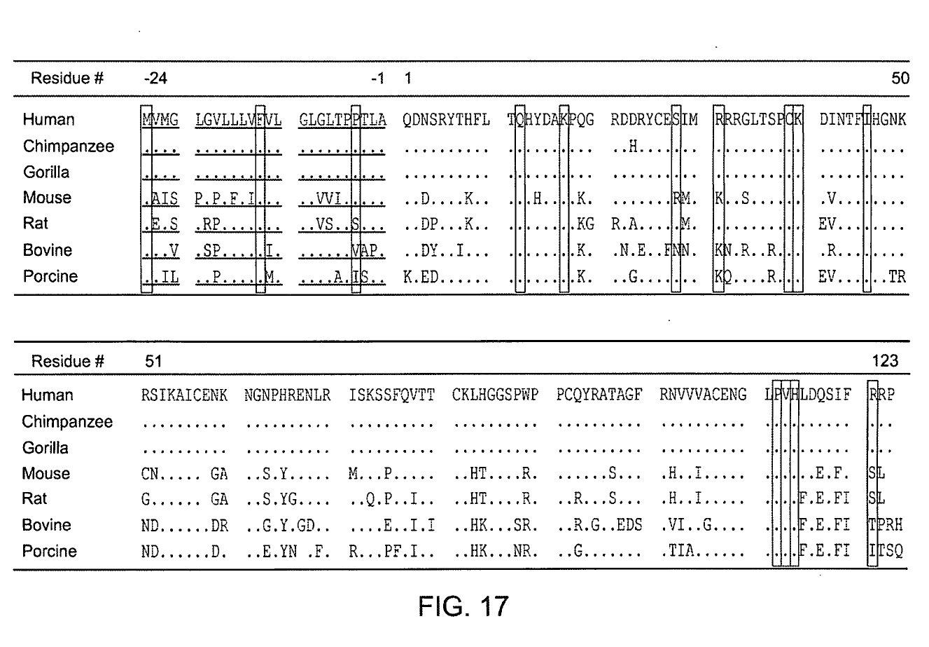

Recently, linkage analysis in Irish and Scottish ALS populations identified an association of the G allele of the single nucleotide polymorphism (SNP) rs11701 in the coding region of ANG (representing the amino acid residue G86 in the mature protein) (Greenway et al. (2004) Neurology 63:1936). Subsequently, seven heterozygous missense mutations in ANG were identified in 15 patients by sequence screening of 1,629 individuals with ALS (Greenway et al. (2006) Nat. Genet. 38:411), an overall frequency of approximately 1% with an over representation of familial ALS (FALS) (4/259, 1.5%) over sporadic (SALS) (11/1370, 0.8%). From sequencing an additional 298 ALS patients of a Northern American cohort, an additional four mutations in the ANG gene were identified (1.3% frequency) (Wu et al. (2007) Ann. Neurol. 62:609). More recently, 7 more mutations were identified in 9 of the 737 Italian ALS patients (Gellera et al. (2007) Neurogenetics). ANG mutations in Italian population also seem to segregate FALS (3/132, 2.3%) from SALS (6/605, 1%) with an overall frequency of 1.2%. Id. FIG. 17 lists the amino acid sequences of primate and mammalian ANG with the mutations bracketed.

So far, a total of 15 missense mutations (at 14 positions) in the coding region of ANG have been identified in 37 of the 4193 ALS patients of the Irish, Scottish, Swedish (Greenway et al. (2006) Nat. Genet. 38:411), North American (Wu et al. (2007) Ann. Neurol. 62:609), Italian (Gellera et al. (2007) Neurogenetics), and French (Paube et al. (2008) Arch. Neurol. 65:1333) populations. Among these mutations, three occurred in the signal peptide regions and 10 in the mature protein. In the six sequencing efforts carried out so far, a total of 3341 healthy controls have been included and two mutations in the ANG gene were found in healthy controls (Greenway et al. (2006) Nat. Genet. 38:411; Gellera et al. (2007) Neurogenetics). The first is a K17I mutation that was found in an apparent healthy 65-year-old male of European descent. The second is the 146V mutation that was found in 11 of the 1568 Italian healthy control patients (Corrado et al. (2007) J. Neurol. Sci. 258:123; Conforti et al. (2007) Neuromuscul. Disord.; Gellera et al. (2007) Neurogenetics; Del Bo et al. (2006) Neurobiol Aging). Therefore, 146V mutation does not seem to be associated with Italian ALS patients but does seem to be associated with the Scottish ALS patients in which 3 of the 398 ALS patients but none of the 299 controls harbor the 146V mutation (Greenway et al. (2006) Nat. Genet. 38:411). Table 1 lists the frequencies of ANG mutations occurred in 3001 ALS patients.

TABLE 1

| Mutation | Number of Cases | References |
|---|---|---|
| M(-24)I | 2 | Conforti et al. (2007) |
| | | Neuromuscul. Disord.; |
| | | Gellera et al. (2007) |
| | | Neurogenetics |
| F(-13)S | 1 | Conforti et al. |
| P(-4)L | 2 | Gellera et al.; |
| | | Wu et al. (2007) Ann. |
| | | Neurol. 62: 609 |
| Q12L | 2 | Greenway et al. (2006) Nat. |
| | | Genet. 38: 411 |
| K17I | 4 | Greenway et al.; |
| | | Wu et al. |
| | | Van Es et al (2009) |
| | | Neurology 71: 287) |
| K17E | 2 | Greenway et al. |
| S28N | 1 | Wu et al. |
| R31K | 1 | Greenway et al. |
| C39K | 2 | Greenway et al. |
| K40I | 3 | Greenway et al. |
| I46V | 10 | Corrado et al. (2007) J. |
| | | Neurol. Sci. 258: 123; |
| | | Greenway et al.; |
| | | Gellera et al. |
| P112L | 1 | Wu et al. |
| V113I | 2 | Gellera et al. |
| H114R | 1 | Gellera et al. |
| R121H | 1 | Paube et al. (2008) Arch. |
| | | Neurol. 65: 1333 |

Two new mutations, D22G and K60E, have been discovered in our ongoing effort to screen more ALS patients.

### Example 4

### Properties of Mutant ANG Proteins

Except for the three mutations in the signal peptide regions (M−241, F-13S, P-4S) and the two in the coding region (V113I, H114R) that were just reported in the most recent publication (Gellera et al. (2007) Neurogenetics), all the mutant ANG proteins have been prepared and characterized by ribonuclease assay (Wu et al. (2007) Ann. Neurol. 62:609; Crabtree et al. (2007) Biochemistry 46:11810), nuclear translocation assay (Wu et al. (2007) Ann. Neurol. 62:609), and angiogenesis assay (Id.). Except for R31K, all of these mutant proteins have severely impaired ribonucleolytic activity ranging from <1% (K40I) to 19% (K17E) of the wild-type ANG. R31K has 69% of the wild-type enzymatic activity (Crabtree et al. (2007) Biochemistry 46:11810). Because the RNase activity of ANG has been proven to be essential for angiogenesis (Shapiro et al. (1986) Biochemistry 25:3527; Shapiro and Vallee (1989) Biochemistry 28:7401), these mutant ANG proteins are likely to be inactive in inducing angiogenesis. Some of them also seem to have a reduced thermal stability (Crabtree et al. (2007) Biochemistry 46:11810). Among the three mutant ANG proteins (K171, S28N, P112L) tested in the nuclear translocation assay, S28N and P112L do not undergo nuclear translocation and K17I has a reduced capacity (Wu et al. (2007) Ann. Neurol. 62:609). Two types of angiogenesis assays have been used to test the angiogenic activity of the mutant ANG proteins. The endothelial cell tube formation assay on fibrin gel was used to examine the mutants identified from the Northern American ALS cohort and the results showed that all three mutants (K17I, S28N, P112L) are inactive. Id. The aorta ring assay was used to test three of the seven mutants identified from the Irish and Scottish ALS populations. All three mutants tested (Q12L, C39W, K40I) were inactive in the aorta ring angiogenesis assay (Crabtree et al. (2007) Biochemistry 46:11810). Taken together, these results demonstrate that ANG mutations identified in ALS patients are associated with a functional loss of the angiogenic activity of the ANG protein.

Wild-type ANG has been shown to induce neurite outgrowth and pathfinding of motor neurons derived from P19 embryonal carcinoma cells (Subramanian and Feng (2007) Hum. Mol. Genet. 16:1445). ANG also protects P19-derived motor neuron from hypoxia-induced cell death, but the ALS-associated mutant ANG proteins (Q12L, C39W, K40I) lack this neuroprotective activity (Subramanian et al. (2008) Hum. Mol. Genet. 17:130). Moreover, these mutant ANG proteins are cytotoxic to the P19-derived motor neurons and induce their degeneration, suggesting that ANG mutations may even be causative to ALS. Id.

### Example 5

### Expression of ANG in the CNS

Mouse ANG is strongly expressed in the developing mouse nervous system both in the brain and in the spinal cord (Subramanian and Feng (2007) Hum. Mol. Genet. 16:1445). Immunohistochemistry (IHC) and immunofluorescence (IF) have been used to show that ANG expression is the strongest in the presumptive forebrain, midbrain, hindbrain and spinal cord at 9.5 day pc. Id. At 11.5 day pc, ANG expression remains high in the telencephalon, mesen and mylencephalon as well as in the spinal cord, spinal ganglia and choroids plexus. Id. Until mid-gestation, ANG expression is stronger in the nervous system than in any other tissues. Co-staining with Peripherin and Islet1 showed that ANG is expressed in mouse motor neurons.

IHC was also used to detect the expression of human ANG in normal spinal cords obtained from fetal (ranging from 15 to 30 weeks gestation) and adult human autopsies. Strong ANG staining was observed in the spinal cord ventral horn motor neurons of both fetal and adult cases (Wu et al. (2007) Ann. Neurol. 62:609). ANG was also detected in the extracellular matrix and interstitial tissues in all cases, consistent with ANG being a secreted protein. It appears that ANG expression in the spinal cord is down-regulated as development proceeds but is still strongly expressed in the adulthood. Strong cytoplasmic and nuclear accumulation of ANG in motor neurons of both prenatal and adult spinal cords suggest a physiological role of ANG, both early in development and later in adulthood, and supports the hypothesis that ANG mutations are likely relevant to ALS pathology.

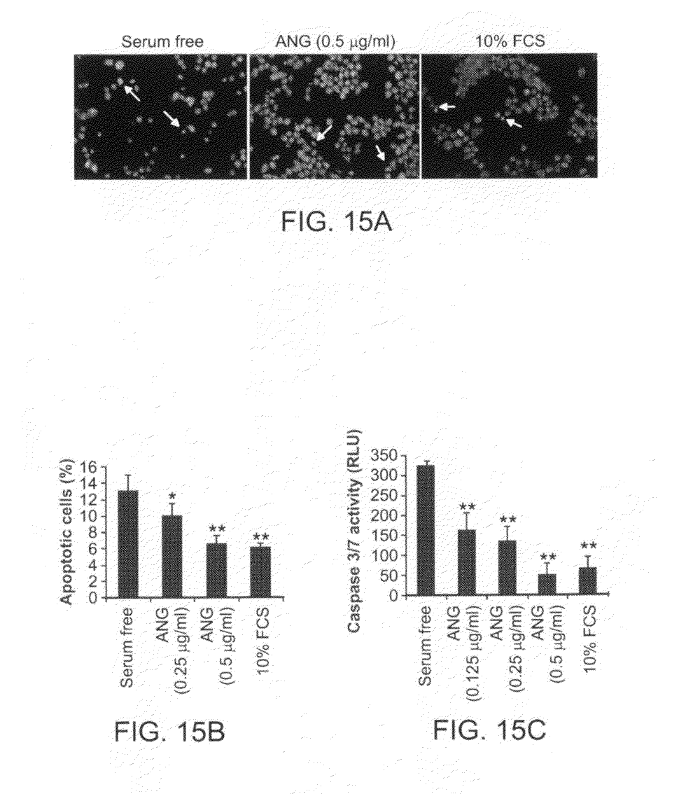

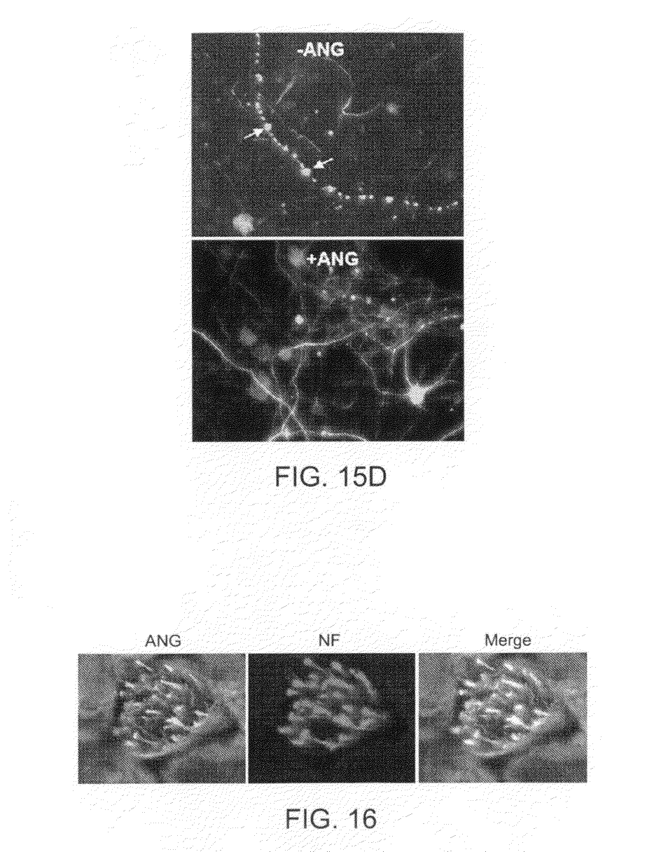

In the developing human brain, strong ANG expression in other types of neural cells such as ependymal cells, purkinje cells and glial cells in the cerebellum (FIG. 15A) and the motor neurons of cranial nerves and substantia nigra (FIG. 15B) have been detected. It is of interest to note that neurons that express ANG strongly are all related to motion.

Double IF with an anti-ANG monoclonal antibody (mAb) and anti-von Willebrand factor (vWF) polyclonal antibody (pAb) showed that ANG is localized in both endothelial cells and motor neurons of spinal cord tissues, suggesting that ANG may also mediate angiogenesis in the spinal cord, and may play a role in maintaining the physiological health of motor neurons. Id. Thus, ANG abnormalities may have a dual role in ALS—directly through motor neuron function and indirectly through endothelial cells and aberrant angiogenesis.

### Example 6

### ANG Protein Level is Decreased in Spinal Cord Motor Neurons of ALS Patients

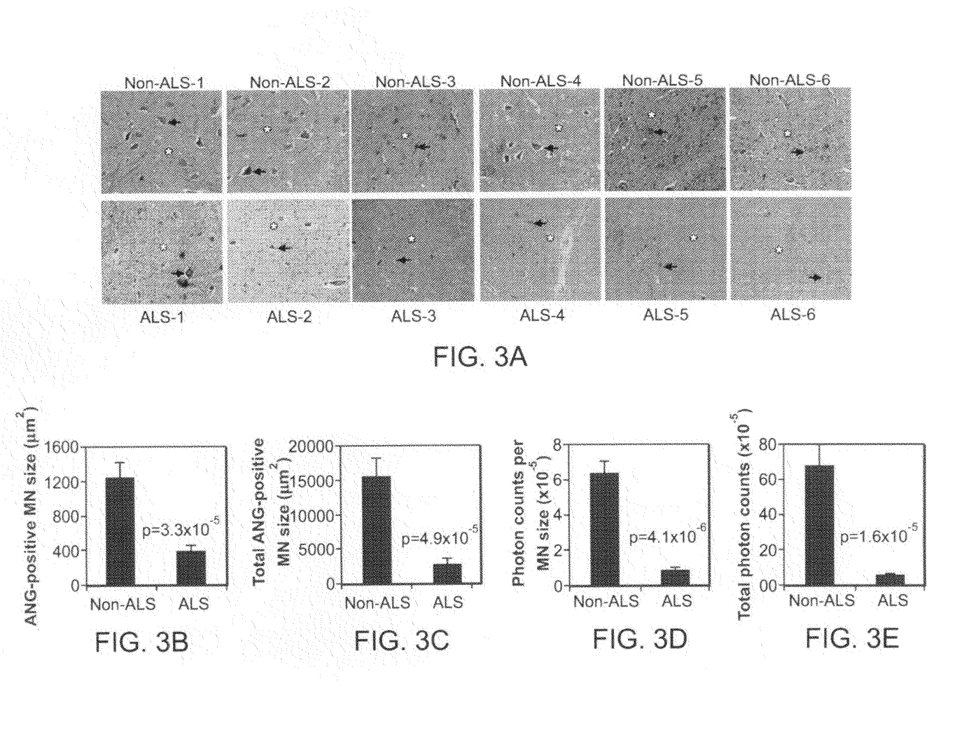

This example demonstrates that ANG expression was decreased in the spinal cord of ALS patients that do not harbor ANG mutations. IHC with the human ANG-specific mAb 26-2F showed that ANG protein level was markedly decreased in both motor neurons (indicated by arrows) and stroma (indicated by stars) of the spinal cord of human ALS patients as compared to that of the non-ALS controls (FIG. 3A). A total of six ALS cases and six non-ALS control spinal cords were examined.

The mAb 26-2F is known to be specific for human ANG. It does not recognize any other human proteins. An isotype-matched, non-immune IgG was used as a negative control and no signals were observed in both ALS and non-ALS specimens under the same concentration. Moreover, an affinity-purified anti-human ANG polyclonal antibody (pAb) R113 was also and the results were the same to that obtained with mAb 26-2F. These data confirm specificity of the IHC and the subsequent IF results.

Imaging quantification was performed with the Image) software. The entire ventral horn was analyzed. The average size of the ANG-positive motor neuron was 1245±170 and 385±63 μm², respectively, in normal and ALS spinal cord (FIG. 3B), representing a 69% decrease in ALS (p<3.3×10⁻⁵). The total area covered by ANG-positive motor neuron in the ventral horn of normal and ALS patients are 15.5±2.6×10³ and 2.8±0.8×10³ μm², respectively (FIG. 3C), representing a 72% decrease in ALS (p<4.9×10⁻⁵). To obtain the ANG staining intensity in motor neurons, the photon counts of the adjacent non-motor neuron area were used as the controls. The average photon counts per motor neuron of normal and ALS spinal cord were 6.3±0.7×10⁵ and 7.8±1.2×10⁴, respectively (FIG. 3D), representing a 78% decrease in ALS (p<4.1×10⁻⁶). The total photon counts (FIG. 3E) in the motor neuron in normal and ALS spinal cord were 6.7±1.2×10⁶ and 5.3±0.9×10⁵, respectively, representing a 92% decrease in ALS (p<1.6×10⁻⁵). These results indicate that ANG protein level in the spinal cord of ALS patients was dramatically decreased.

### Example 7

### ANG Protein Level was Decreased in Both Motor Neurons and Endothelial Cells of ALS Spinal Cord

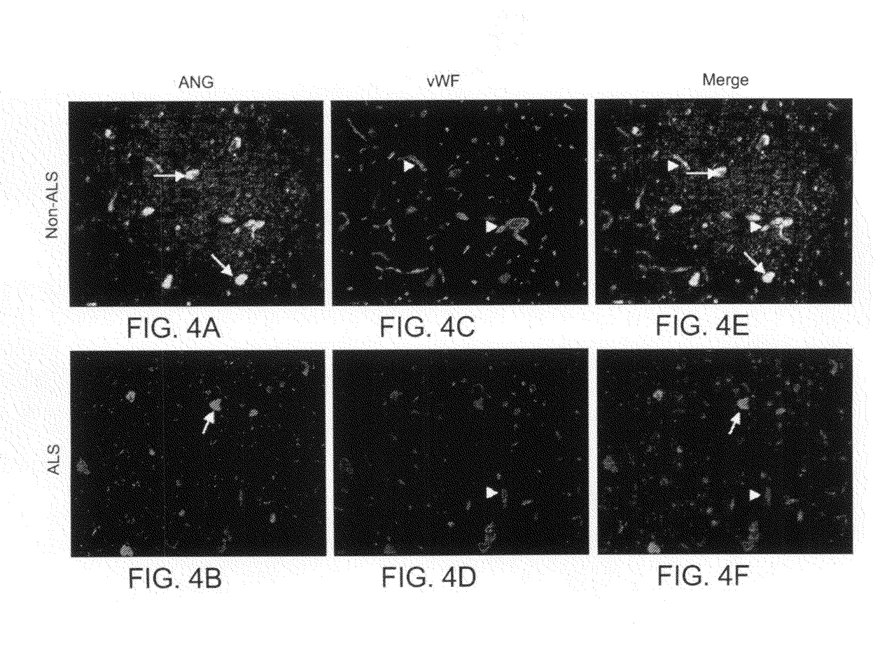

Double IF staining with 26-2F (FIGS. 4A-B) and anti-von Willebrand factor (vWF) (FIGS. 4C-D) was carried out to detect ANG and blood vessels, respectively. The merged images of ANG and vWF staining shows that ANG is located both in motor neurons (indicated by arrows) and in blood vessels (indicated by arrow heads) and its level in both cell types is decreased in the spinal cord of ALS patients (FIGS. 4E-F).

### Example 8

### ANG mRNA Levels Decreased in ALS Spinal Cord

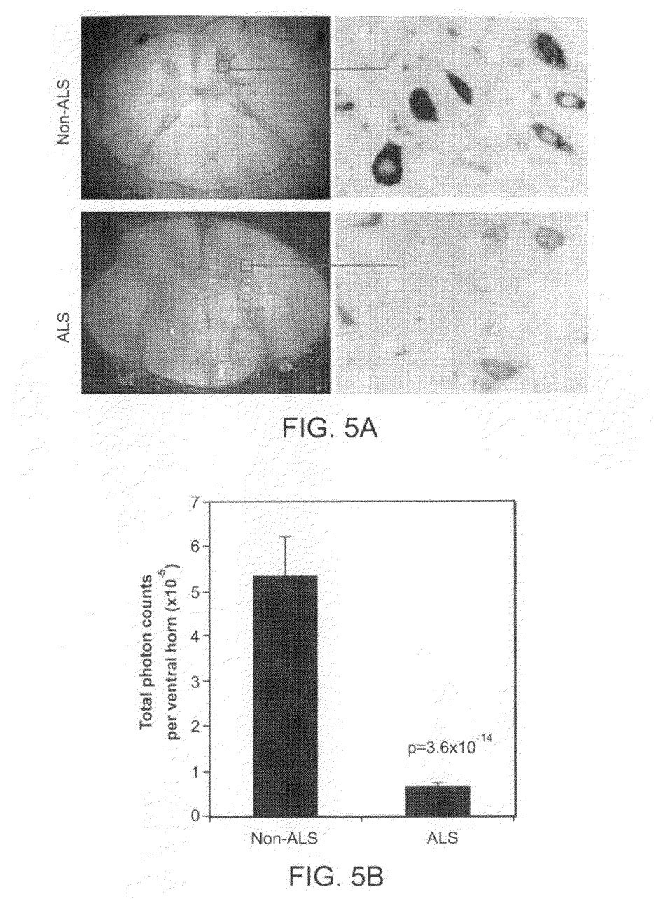

In situ hybridization (ISH) was used to check the mRNA level of ANG in the ALS and control spinal cords. Strong staining of ANG mRNA in the motor neurons was observed in non-ALS spinal cord, which was dramatically decreased in ALS spinal cords (FIG. 5A). Quantitative analysis of the ISH images indicate that the total photon counts of ANG mRNA staining in the motor neurons of the entire ventral horn of ALS and non-ALS spinal cord were 5.3±0.8×10⁵ and 6.4±0.8×10⁵, respectively (FIG. 3E), representing an 88% decrease in ALS samples.

The results shown in FIGS. 3-5 indicate that ANG is strongly expressed in normal human spinal cords and that its expression is decreased in ALS patients. At present, it is unknown whether decreased ANG level in the spinal cord of ALS patients is a cause or a consequence of motor neuron degeneration. However, these findings indicate that a deficiency in ANG activity might be related to ALS pathogenesis. These results also indicate that local production of ANG may be more relevant to ALS. Without intending to be bound by scientific theory, transcription from the distal promoter (i.e., non-liver) may be affected in ALS patients differently from transcription from the proximal promoter (i.e., liver-specific).

### Example 9

### Protein and mRNA Levels of Mouse Angiogenin (ANG) is Decreased in the Spinal Cord of SOD1^{G93A} Mice

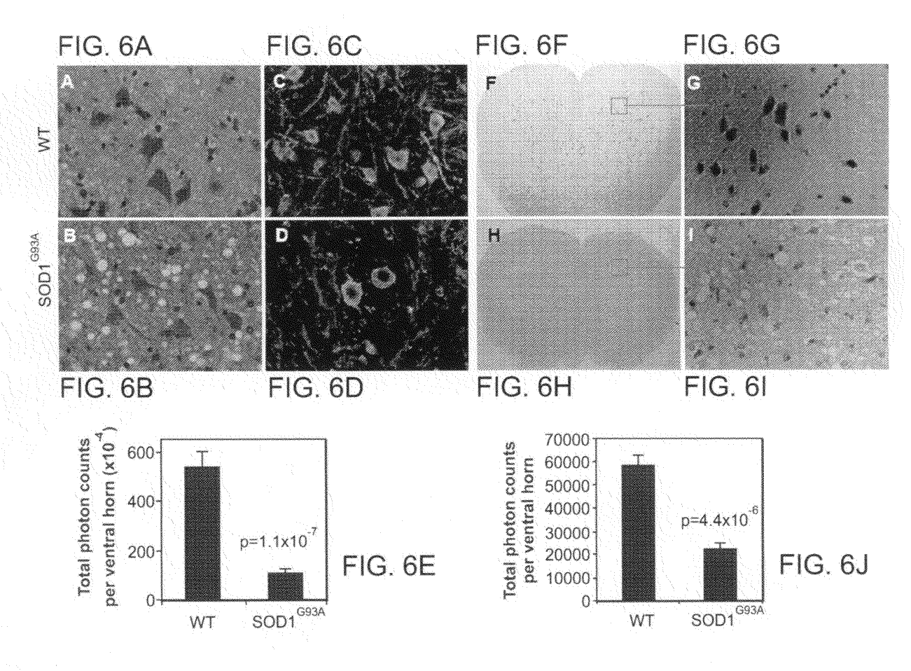

Transgenic mice over-expressing the G93A mutant SOD1 gene develop symptoms mimicking that of human ALS patients and are an established model for ALS research (Gurney (1997) J. Neurol Sci. 152 Suppl. 1:S67). INC, IF and Western blotting were used to determine the protein level and use ISH for the mRNA level of mouse ANG in the spinal cord of WT and SOD1^{G93A} mice. Animals were sacrificed at 14 weeks of age after transcardiac perfusion and the spinal cords were processed for IHC (FIG. 6A-B) and IF (FIG. 6C-D) with an anti-mouse ANG IgG R165.

Mouse ANG protein levels in the ventral horn motor neurons of SOD1^{G93A} spinal cord were much lower than that of WT mice at the same age. The antibody used in these experiments was prepared using recombinant mouse ANG 1 and was affinity-purified. While humans have only one ANG gene, mice have 6 ANG isoforms. It is not known at present whether R165 recognizes the other ANG isoforms, but ANG1 was the predominant isoform expressed in the spinal cord. The signals in FIGS. 6A-D were most likely from ANG1. No IHC or IH signals were detected when a non-immune rabbit IgG was used under the same conditions (30 μg/ml IgG). Quantitative image analysis indicated that the total photon counts of mouse ANG in SOD 1^{G93A} and WT mice were 1.1±0.2×10⁶ and 5.4±0.7×10⁶, respectively, representing an 80% decrease (p=1.1×1e) in SOD1^{G93A} mice (FIG. 6E).

Next, ISH was performed using a riboprobe specific for ANG1 to detect the mRNA level of ANG1 (FIGS. 6F-I). ANG1 mRNA levels in the spinal cord of SOD1^{G93A} mice (FIGS. 6H-I) was significantly lower than that of the WT (FIGS. 6F-G). Quantitative image analyses indicated that the photon counts in SOD1^{G93A} and WT mice were 5.9±0.4×10⁴ and 2.2±0.2×10⁴, respectively (FIG. 6J), representing a decrease of 63% (p=4.4×10⁻⁶). Therefore, both ANG1 protein and mRNA levels were decreased in the motor neurons in SOD1^{G93A} mice. It is currently unknown how SOD1^{G93A} expression results in the decrease of ANG expression in the spinal cord motor neurons.

### Example 10

### Decreased Blood Vessel Size in Spinal Cords of Human ALS Patients and SOD1^{G93A} Mice

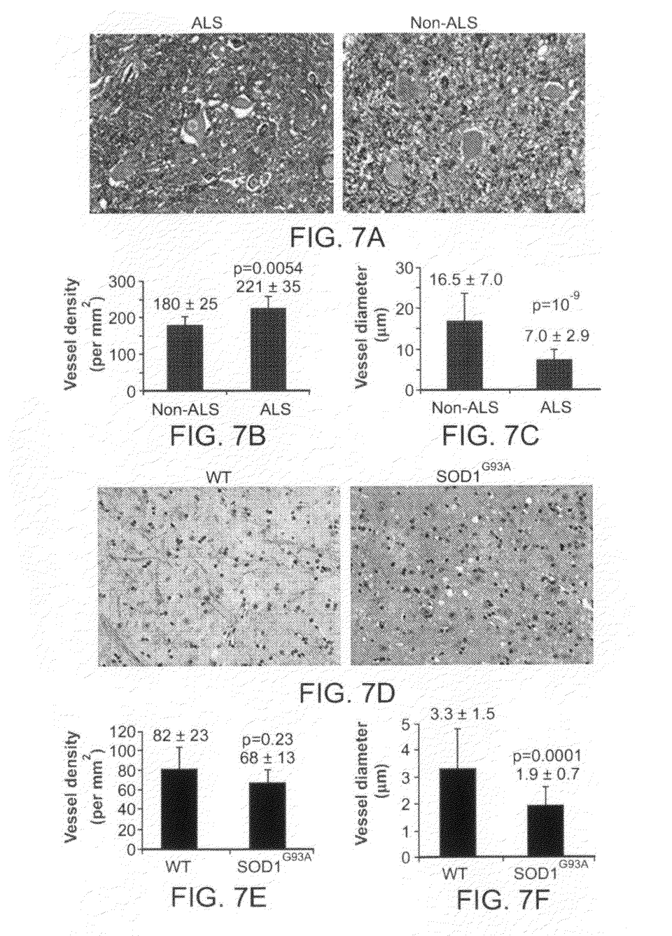

The density and size of the blood vessels in control and ALS spinal cords were investigated. IHC with an anti-vWF antibody was used to stain the blood vessels (FIG. 7A). The numbers of the vessels in five randomly selected areas from each of the two ventral horns (n=5) were counted and their sizes measured. FIG. 7B shows that the density of blood vessels in the ALS spinal cord was marginally but statistically significantly increased (p=0.0054). However, the average size of the vessel was dramatically decreased (p=1×10⁻⁹) (FIG. 7C). Similarly, the vessel density in the SOD1^{G93A} and WT mice was not much different (p=0.23) (FIG. 7E), but the vessel size was significantly decreased (FIG. 7F, p=0.0001). These results indicate that motor neuron degeneration may be associated with an irregular vasculature in the spinal cord.

### Example 11

### Intraperitoneally Injected ANG Protein Reaches at Spinal Cord

Without intending to be bound by scientific theory, one of the reasons for a relative minimal effect of exogenous trophic factors and other types of therapeutic proteins could be their failure to cross the blood brain barrier (BBB) and blood spinal cord barrier (BSCB). Gene therapy is therefore an alternative approach for ALS therapy. Many strategies are under investigation, including the delivery of genes encoding neurotrophic factors, anti-apoptotic drugs and antioxidants using viral vectors administered directly into the affected areas of the central nervous system (CNS), or through retrograde transport to motor neurons from intramuscular injection, or through ex vivo gene transfer (Federici and Boulis (2006) Muscle Nerve 33:302). Besides lentiviral vector-mediated delivery of VEGF (Azzouz et al. (2004) Nature 429:413), AAV-mediated delivery of IGF-1 (Kaspar et al. (2003) Science 301:839), GDNF (Wang et al. (2002) J. Neurosci. 22:6920), and Bcl-2 (Azzouz et al. (2000) Hum. Mol. Genet. 9:803) genes have also been shown to be effective in the SOD1^{G93A} transgenic mice.

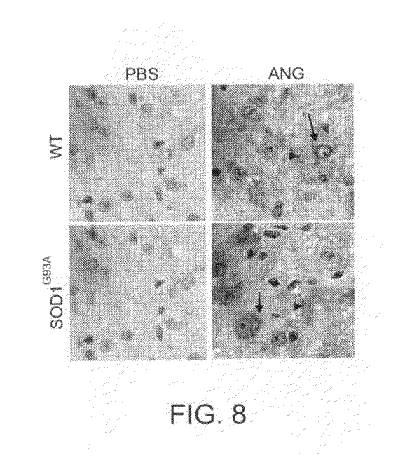

To ascertain the feasibility of systemic administration of ANG protein in potential ALS treatment, it was determined whether i.p.-injected ANG could reach the spinal cord. For this purpose, WT and SOD1^{G93A} mice, 11 weeks of age, were injected with 10 μl of PBS or 10 μg of human WT ANG. IHC with the human ANG-specific mAb 26-2F showed that ANG reached the spinal cord (FIG. 8) of both WT and SOD1^{G93A} mice. As the mice were transcardiac perfused with 30 ml PBS before sacrifice, it was unlikely that the spinal cord samples were contaminated with the human ANG that entered circulation after i.p.-injection. Moreover, the same amount of EGF, bFGF, and RNase A were injected in the same manner, and no significant amount of these proteins was detected in the spinal cord (data not shown). RNase A has a 56% homology to ANG at the amino acid level (Strydom et al (1986) Biochemistry 25:3527), has the same molecular weight, similar pI, and a very similar 3-D structure (Acharya et al et al. (1991) PNAS 92:2049), but is not angiogenic. The unique property of i.p.-injected ANG protein crosses the BBB or BSCB allowed the systemic administration of ANG protein to assess its therapeutic activity toward ALS in SOD1^{G93A} mice.

### Example 12

### ANG Treatment Improved Motor Muscular Function and Survival of SOD1^{G93A} Mice

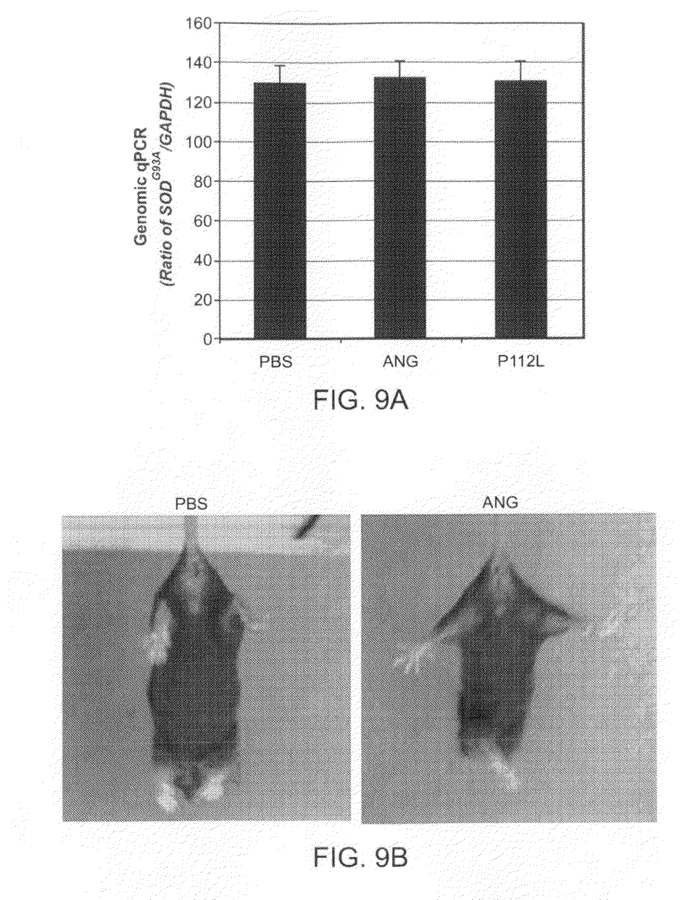

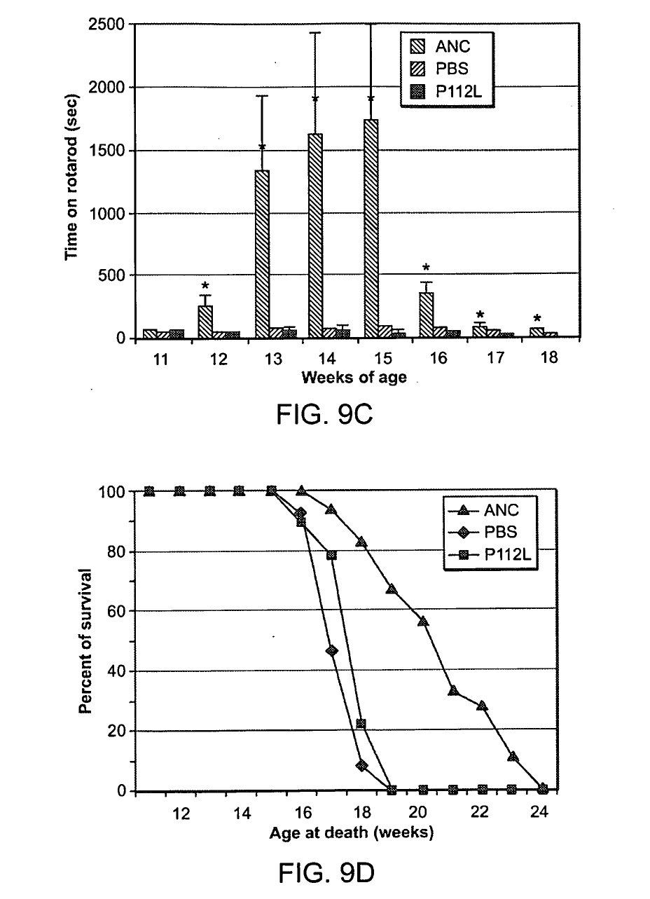

The effect of ANG on motor muscular function of SOD1^{G93A} mice was investigated. A total of 36 SOD1^{G93A} mice were separated into three groups. Each group had 12 litter- and gender-matched mice. Quantitative PCR was performed on every SOD1^{G93A} mouse to ensure that a relatively equal copy number of the transgene was present in each mouse (FIG. 9A).

Eleven week old mice were treated with weekly i.p. injections of PBS, WT or the P112L mutant ANG protein at 10 μg per mouse, and their motor muscular function was tested on a rotarod in a blinded fashion. FIG. 9B shows that ANG-treated mice were able to extend their hind legs when lifted by the tail, but the PBS-treated mice failed to do so, indicating that the muscle strength was enhanced by ANG treatment. To compare the effect of P112L mutant with WT ANG, IHC was performed with the mAb 26-2F to ensure that i.p.-injected P112L mutant protein indeed reached spinal cord as did WT protein. Rotarod performance showed that ANG treatment dramatically increased the time the animals could stay on the rotarod as compared to the PBS and P112 treatments (FIG. 9C), indicating that i.p. ANG treatment enhanced the motor coordination and muscular function of the SOD1^{G93A} mice. Student's 1-test indicated that the difference in the rotarod time was highly significant from week 12 to 18 (p<0.0001 in all these 7 weeks, indicated by *). At the peak performance (at week 14), ANG-treated mice were able to stay on the rotarod for an average of 1726±778 sec, a 32-fold increase over that of the PBS-treated control mice (53±3 sec) (FIG. 9C). At the same week, P112L ANG-treated mice were able to stay on the rotarod for an average time of 51±21 sec.

ANG treatment increased the survival of these animals by four weeks (FIG. 9D), representing a 23% increase in the life span of these mice. These results indicate that ANG treatment may prolong the life of ALS patients as well as improving quality of life. In certain exemplary embodiments, an increased life expectancy of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or more is obtained. It is important to note that the P112L mutant protein had no beneficial effect on SOD1^{G93A} mice on either the rotarod test or the tail suspension test, and did not prolong their survival. These results demonstrate that the beneficial effect of ANG on SOD1^{G93A} mice is dependent on the angiogenic activity. They also support the hypothesis that ANG mutations are associated with ALS pathogenesis. ANG had no detectable effects on WT mice at the same dose on rotarod test. No side effects in animal grooming behavior were observed.

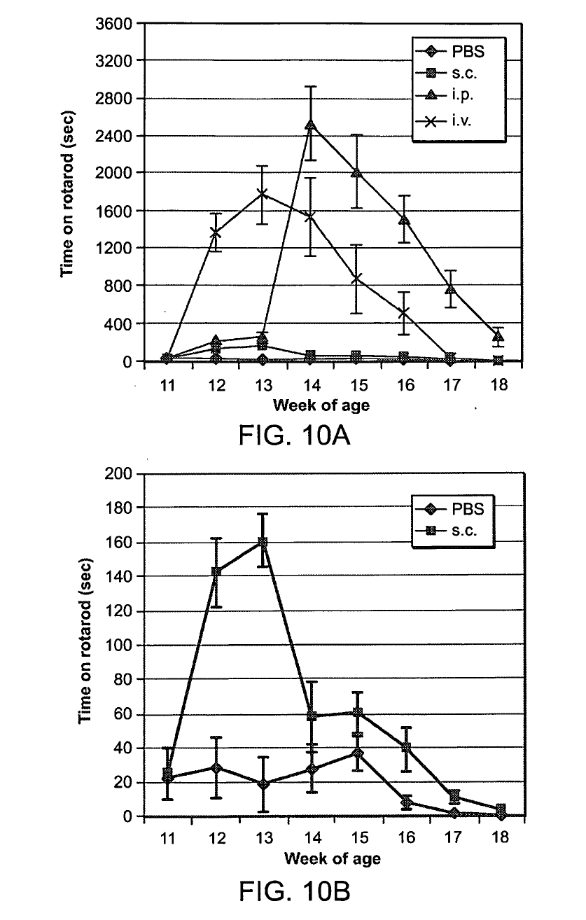

The efficacy of different routes of ANG administration including intravenous (i.v.), subcutaneous (s.c.) and i.p. at the same dose (10 μg per mouse) and same interval (every week) were compared. All three routes of administration were effective in increasing the performance on rotarod with the descending order of i.v.>i.p.>s.c. (FIG. 6). For example, at week 13 (after 2 injections), the time the mice were able to stay on a rotating rotarod were 161±15, 259±27, and 1770±310 sec, respectively, for the s.c., i.p., and i.v. ANG treatment, respectively. All three routes of ANG administration show significantly better performance on the rotarod as compared to the untreated group (19±16 sec). Intravenous injection provides the fastest improvement in motor muscular function (FIG. 10). The maximal effect of i.v. and i.p. injection appeared at week 13 and 14, respectively. Please note the different scales used in the two panels in order to reveal the difference in the beneficial effect of 3 routes of administration. Among the three routes of administrations, s.c. injection was the least effective. However, the beneficial effect of s.c. injection of angiogenin in improving motor muscular function of SOD^{G93A} is still better than that mediated by IGF and VEGF reported in prior art (Gage et al. (2003), Science 301, 839; Azzouz et al. (2004) Nature 429, 413).

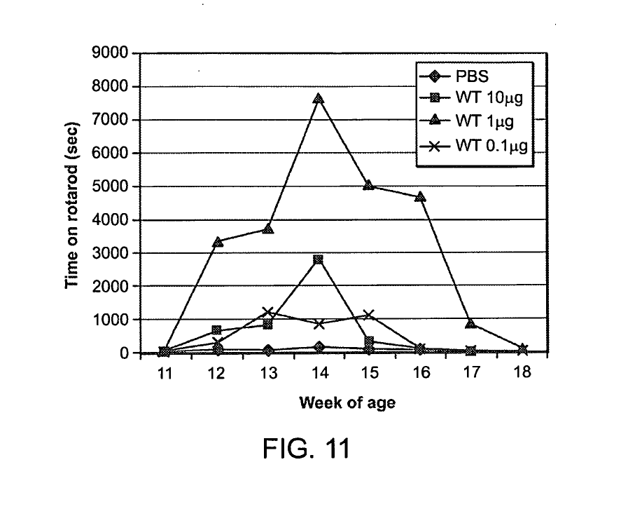

In order to determine the optimal dose of angiogenin in improving the motor muscular function of SOD^{G93A} mice, we compared the effect of i.p injection of ANG protein at 0.1, 1, and 10 μg per mouse per week. As shown in FIG. 11, 1 μg per mouse per week was the most effective dose, while all three doses significantly improved the motor muscular function of SOD^{G93A} mice as determined by their performance on a rotarod.

### Example 13

### ANG Treatment Improved Motor Neuron Survival and Normalized Spinal Cord Vasculature

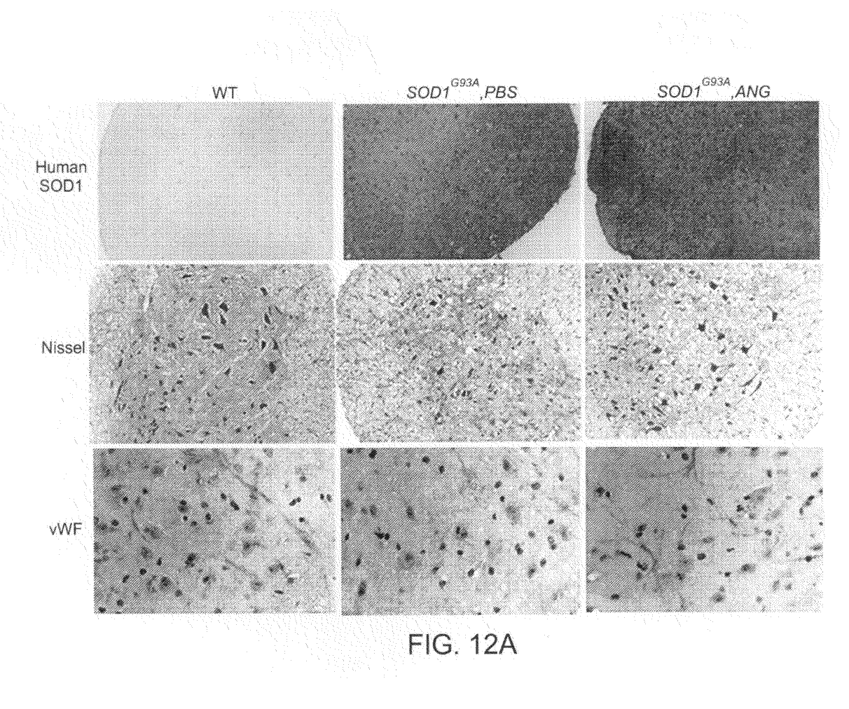

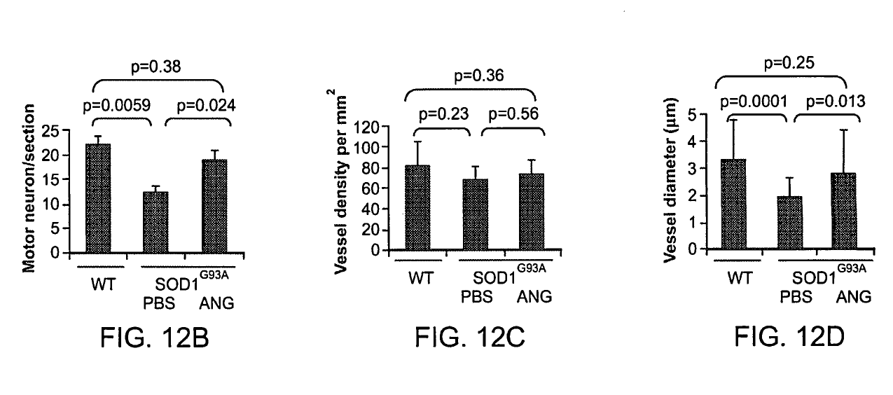

WT mice and SOD1^{G93A} mice treated with PBS or ANG protein were sacrificed at week 15, and the lumbar regions of the spinal cord was removed and processed for histology and IHC. First, an anti-human SOD1 IgG was used to ensure that the transgene expression was not interfered with ANG treatment (FIG. 12A, top panels). Nissl staining showed that there was a clear loss of motor neurons in the spinal cord of PBS-treated SOD1^{G93A} mice compared to those of WT mice (FIG. 12A, middle panels). ANG treatment clearly increased the number of motor neurons (FIG. 12B). The number of the motor neurons with a soma size larger than 250 μm² was 21.9±2.1, 12.3±1.3, and 18.7±2.3, respectively, in WT, PBS- and ANG-treated SOD1^{G93A} mice. ANG treatment thus significantly enhanced motor neuron survival (p=0.024).

IHC with an anti-vWF antibody was performed (FIG. 12A, bottom panel) and the density and size of the blood vessels determined. There were no significant differences in the density of the blood vessels between WT (82±23/mm²), PBS- (68±13/mm²) and ANG-treated SOD1^{G93A} (73±14/mm²) mice (FIG. 12C). However, ANG treatment significantly increased the size of the blood vessels in SOD1^{G93A} mouse spinal cords. The average diameter of the WT mouse, PBS- and ANG-treated SOD1^{G93A} mice were 3.3±1.5, 1.9±0.7, and 2.8±1.6 μm, respectively (FIG. 12D). Without intending to be bound by scientific theory, it is possible that ANG treatment improves the motor muscular function through enhancement of motor neuron survival as well as normalization of spinal cord vasculature.

### Example 14

### ANG Stimulates P19 Cell Proliferation and Differentiation

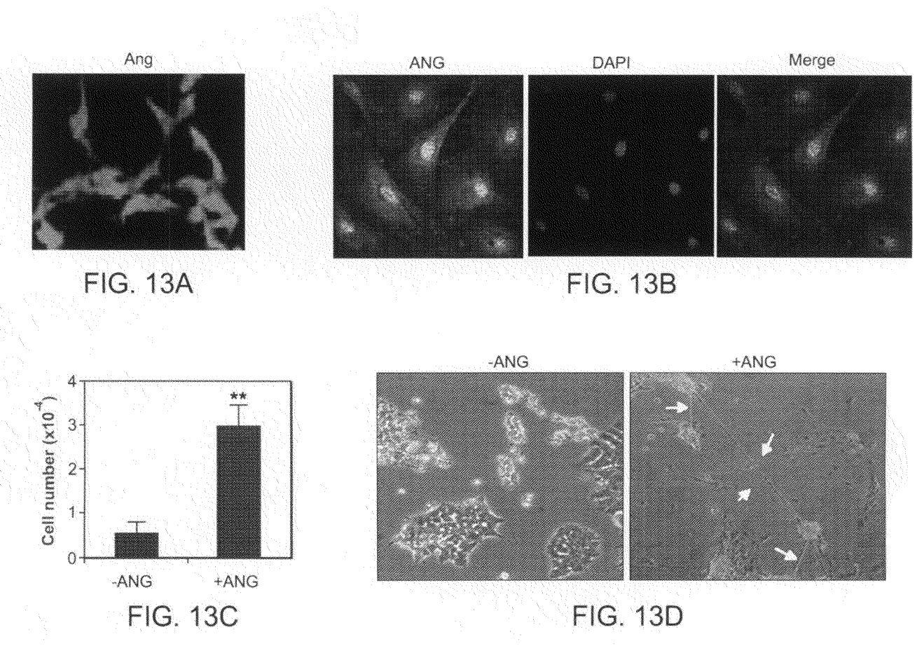

P19 cells are mouse pluripotent embryonal carcinoma cells that have the stem cell-like property of having the ability to both self-renew and differentiate into various types of neural cells (Bain et al. (1994) Bioessays 16:343; McBurney and Rogers (1982) Dev. Biol. 89:503). FIG. 13A shows that undifferentiated P19 cells were stained positive with mouse ANG-specific pAb R165. The role of endogenous ANG in P19 cells is currently unknown. However, human ANG, when added exogenously, underwent nuclear translocation in P19 cells (FIG. 13B) and stimulated the formation of embryoid bodies, the undifferentiated suspended aggregates (FIG. 13C), indicating that ANG stimulates P19 cell proliferation. If these embryoid bodies were resuspended and continued to culture, long neuronal processes formed in the presence of ANG but not in its absence (FIG. 13D), indicating that ANG also induces cell differentiation. These results support a role of ANG in neurogenesis.

### Example 15

### ANG Undergoes Nuclear Translocation in NSC-34 Motor Neuron Cells and Enhances Neurite Outgrowth

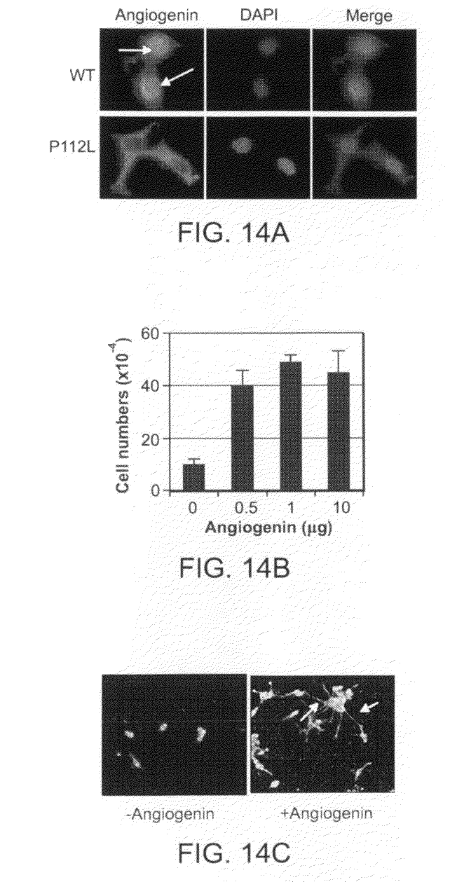

NSC-34 cells are neuroblastoma-spinal cord hybrid cells derived from somatic cell fusion between neuroblastoma N18TG2 cells and motor neuron-enriched embryonic day 12-14 spinal cord cells (Cashman et al. (1992) Dev. Dyn. 194:209). NSC-34 cells display motor neuron like characteristics including generation of action potentials, expression of NF triplet proteins, and acetylcholine synthesis, storage, and release (Matusica et al. (2007) J. Neurosci. Res.; Durham et al. (1993) Neurotoxicology 14:387). Exogenously added human angiogenin was found to undergo nuclear translocation in NSC-34 cells, whereas the P112L mutant angiogenin failed to do so (FIG. 14A). This is consistent with a role of angiogenin in stimulation of rRNA transcription. Angiogenin induced NSC-34 cell proliferation in a dose-dependent manner (FIG. 14B). Moreover, it also promoted neurite outgrowth of NSC-34 cells (FIG. 14C).

### Example 16

### ANG Prevented Stress-Induced Apoptosis of P19 Cells and Embryonic Mouse Cortical Neurons

Serum withdrawal induced P19 cells apoptosis (FIG. 15A, left) that could be prevented by addition of ANG protein (FIG. 15A, middle). In this experiment, apoptotic cells were stained red by ethidium bromide (indicated by arrows), whereas non-apoptotic cells were stained green by acridine orange. FIG. 15B shows that the protective activity of ANG was dose-dependent. Caspase-3 and -7 activities were measured using a luminescent assay, and the results shown in FIG. 15C indicate that ANG prevented caspase 3/7 activation in a dose-dependent manner. In order to know whether ANG protect stress-induced neuron degeneration, mouse embryonic cortical neurons were isolated and cultured for 10 days, and then subjected to hypothermia treatment at 20° C. for 40 min in the absence or presence of 0.5 μg/ml ANG. Neurofilament staining showed that significant fragmentation of the neuronal processing occurred in the absence of ANG but not in the presence of ANG (FIG. 15D). These results indicate that ANG is able to protect stress-induced neuron degenerations.

### Example 17

### Detection of ANG at the Neuromuscular Junction (NMJ)

Double IF was performed to detect whether ANG was located at the NMJ. FIG. 16 demonstrates that in mouse tibialis muscle, ANG (green) and neurofilament (red) was colocalized at the NMJ.

### Example 18

### Angiogenin Variants Having Enhanced Angiogenic Activity

The amino acid sequence of human angiogenin including the nuclear localization sequence ³⁰MRRRG³⁴ is shown in FIG. 17. Three variants of human angiogenin having the following altered nuclear localization sequences were prepared using the procedure described in Shapiro et. al. Analytical Biochemistry 175, 450-461 (1988) incorporated herein by reference: ³⁰KRRRG³⁴ (M30K), ³⁰MRRRK³⁴ (G34K), and ³⁰KRRRK³⁴ (M30KG34K), respectively.

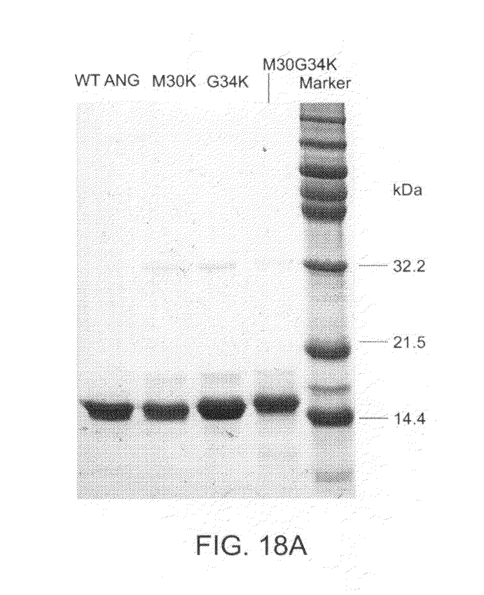

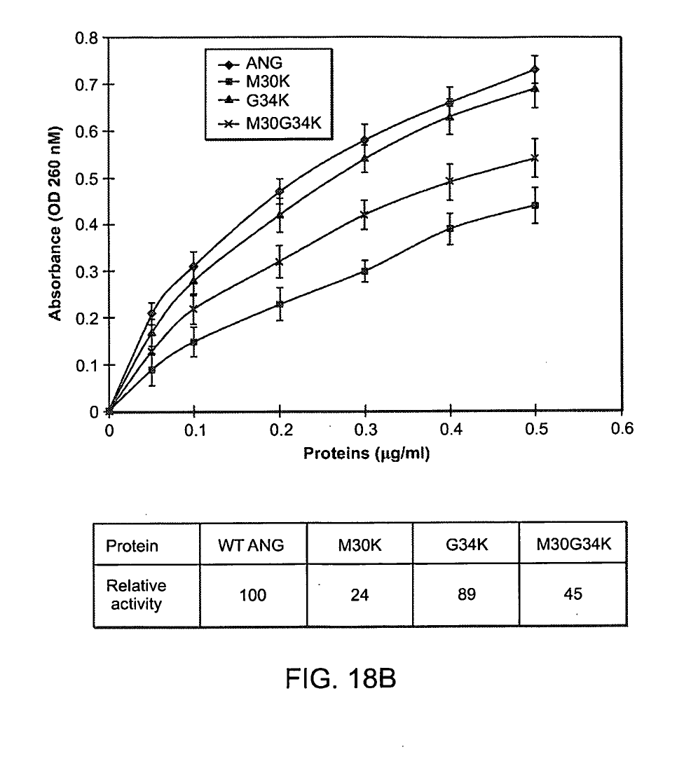

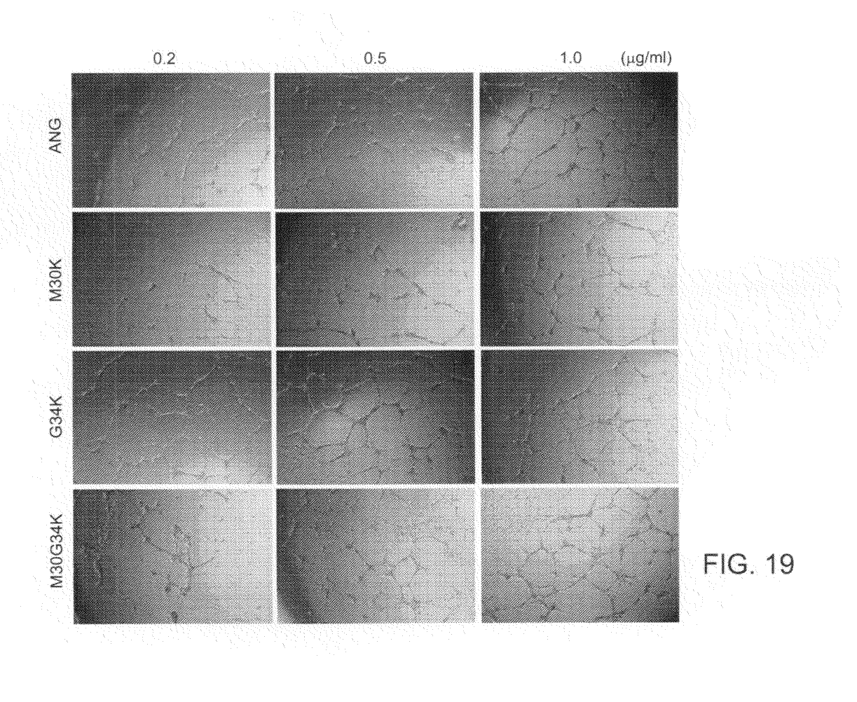

FIG. 18A shows the purity of the recombinant WT and variants by SDA-PAGE and Comassie blue staining. The ribonucleolytic activity of the three variants as compared to the recombinant wild type was determined by the yeast tRNA assay according to Shapiro et al., Biochemistry 25, 3527-32 (1986) incorporated herein by reference and the results shown in FIG. 18B.

The table lists the relative ribonucleolytic activity of the variants as compared to that of wild type angiogenin (100%). The ribonucleolytic activity of M30K, G34K, and M30KG34K variants was 24, 89, and 45%, respectively, of that of wild type angiogenin. Mutation at G34K affects the enzymatic activity of angiogenin only marginally.

The angiogenic activity of the wild type and the three variant angiogenins was examined by Matrigel tube formation assay using the method of Monti et al. (2009), FEBS J., 276:4077 hereby incorporated by reference in its entirety. Human umbilical vein endothelial cells (HUVEC) were cultured on Matrigel coated wells and incubated with WT ANG and variants at various concentrations. Pictures were taken 4 h after incubation.

FIG. 19 shows that G34K angiogenin has enhanced angiogenic activity, whereas M30K and M30KG34K angiogenin have reduced angiogenic activity as compared to that of wild type angiogenin.

According to the experimental results above, G34K has a similar ribonucleolytic activity to that of WT but has an enhanced angiogenic activity.

### Example 19

### Angiogenin Variants Protect P-19 Derived Motor Neurons

P19 cells are mouse pluripotent embryonal carcinoma cells that have the stem cell-like property of having the ability to both self-renew and differentiate into various types of neural cells. Wild type angiogenin has been shown to stimulate neurite outgrowth and pathfinding, and protect stress-induced death of P19-derived motor neurons.

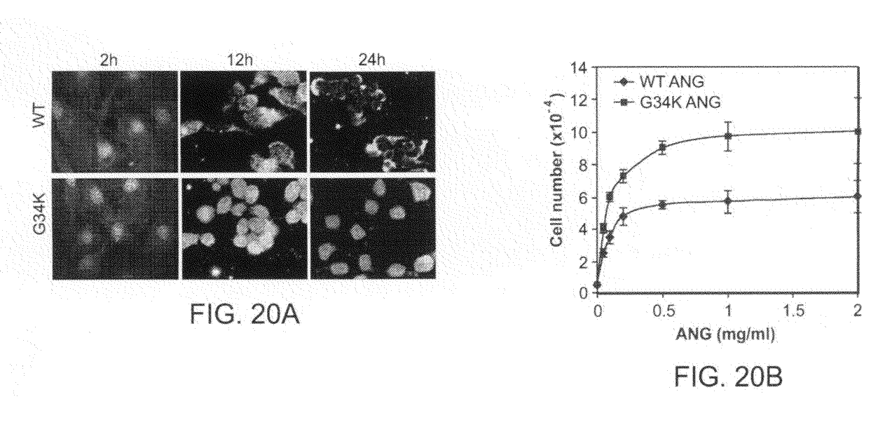

According to the procedure of Subramanian et al. (2008) Human Mol. Genet. 17:130 hereby incorporated by reference in its entirety, P19 cells were incubated with wild type angiogenin and G34K angiogenin for different times and detected by IF with 26-2F. As shown in FIG. 20A, the G34K angiogenin variant was retained in the nucleus for at least 24 hours, whereas most of the wild type angiogenin disappeared from the nucleus in 12 hours.

According to the procedure of Subramanian et al. (2008) Human Mol. Genet. 17:130, P19 cells were incubated with different concentration of wild type angiogenin and the G34K angiogenin variant for 4 days. Cell numbers were determined by a Coulter counter. As shown in FIG. 20B, the G34K angiogenin variant was shown to have higher in vitro proliferative activity toward P19 cells than that of wild type angiogenin.

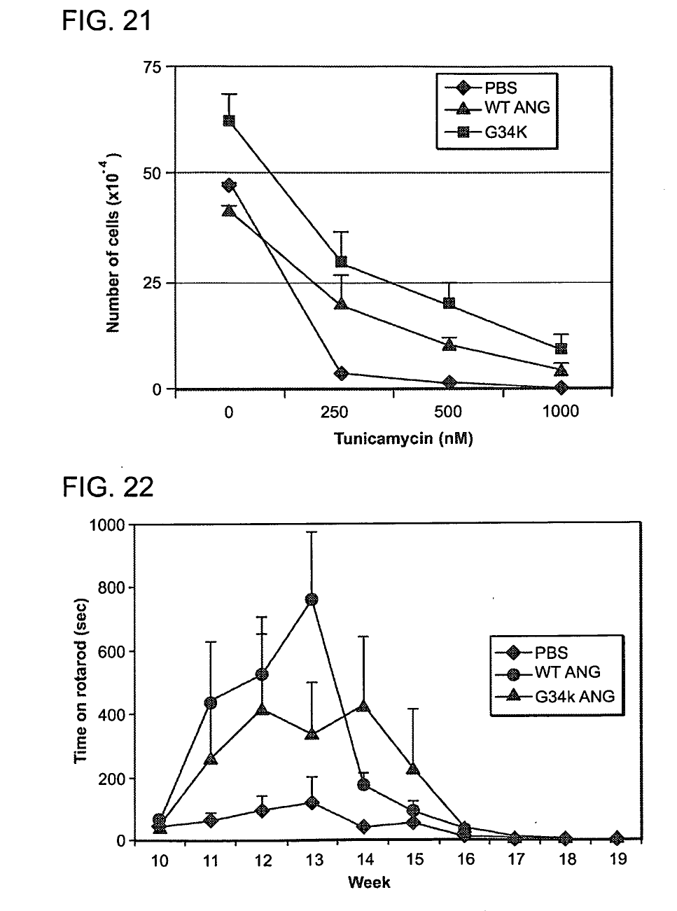

Using the procedure of Subramanian et al. (2008) Human Mol. Genet. 17:130, the neuroprotective activity of the G34K angiogenin variant was also examined with P19 cells under tunicamycin-induced ER stress. P19 cells were treated with tunicamycin at various concentrations in the presence of 400 ng/ml wild type angiogenin or the G34K angiogenin variant for 24 hours. Live cells were determined with Trypan blue and counted. As shown in FIG. 21, tunicamycin-induced cell death was ameliorated by both the wild type angiogenin and the G34K angiogenin variant, but the protective activity of G34K (square) is higher than that of the wild type angiogenin (triangle).

### Example 20

### Angiogenin Variants Enhance the Motor Muscular Function of SOD1^{G93A} Mice

The therapeutic activity of wild type angiogenin and the G34K angiogenin variant in SOD1^{G93A} mice was studied. Ten-week-old mice were treated with weekly i.p. injections of PBS, wild type or the G34K angiogenin variant at 10 μg per mouse, and their motor muscular function was tested on a rotarod in a blinded fashion. FIG. 22 shows that both wild type and the G34K angiogenin variant were able to significantly enhance the motor muscular function of SOD1^{G93A} mice.

### Example 21

### Angiogenin and Angiogenin Variants Prevent Weight Loss in SOD1^{693A} Mice

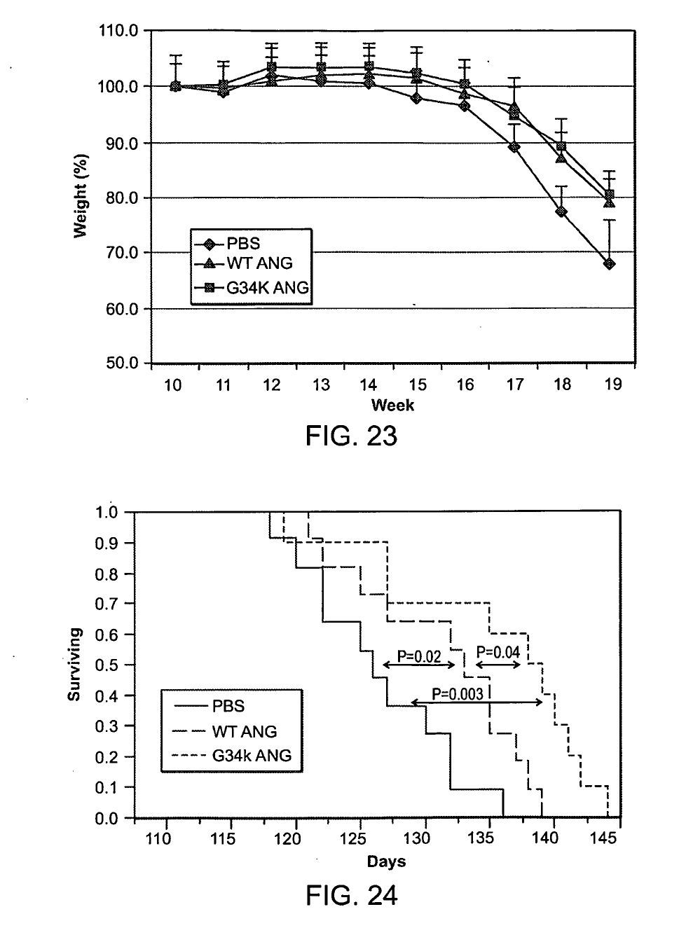

Consistently, both wild type angiogenin and the G34K angiogenin variant prevent weight loss of SOD1^{G93A} mice. As shown in FIG. 23, Student t-test indicated that the body weight measured weekly of the survived animals treated with either wild type or the G34K angiogenin variant were significantly higher than that of the PBS control group from week 15 to 19.

### Example 22

### Angiogenin and Angiogenin Variants Prolong Survival of SOD1^{G93A} Mice

Both wild type angiogenin and the G34K angiogenin variant prolonged the survival of SOD1^{G93A} mice. Ten-week-old mice were ip-injected weekly with PBS, wild type angiogenin, or the G34K angiogenin variant and analyzed for probability of survival by Kaplan-Meyer plot. As shown in FIG. 24, the median survival for mice treated with PBS, wild type angiogenin, and the G34K angiogenin variant was 125, 134, and 139 days, respectively. Wilcoxon test shows that the p values between PBS and wild type angiogenin, and between PBS and the G34K angiogenin variant was 0.02 and 0.003, respectively, indicating that both wild type angiogenin and the G34K angiogenin variant significantly prolonged the survival of SOD1^{G93A} mice. Moreover, the p value between wild type angiogenin and the G34K angiogenin variant was 0.04, indicating that the G34K angiogenin variant has significantly higher therapeutic activity than that of wild type angiogenin.

1. A method of therapeutically treating a neurodegenerative disorder in a subject in need thereof comprising:
administering to the subject a therapeutically effective amount of a composition comprising an angiogenin variant having a modified nuclear localization sequence;
allowing the angiogenin variant having a modified nuclear localization sequence to pass through one or both of the blood brain barrier and the blood spinal cord barrier; and
reducing one or more symptoms of the neurodegenerative disorder in the subject such that the neurodegenerative disorder is therapeutically treated.

2. The method of claim 1, further comprising allowing nuclear translocation of the angiogenin variant having a modified nuclear localization sequence.

3. The method of claim 1, further comprising allowing the angiogenin variant having a modified nuclear localization sequence to stimulate ribosomal RNA transcription.

4. The method of claim 1, further comprising allowing the angiogenin variant having a modified nuclear localization sequence to stimulate ribosomal biogenesis.

5. The method of claim 1, further comprising allowing the angiogenin variant having a modified nuclear localization sequence to stimulate cell proliferation.

6. The method of claim 5, wherein the cell is one or both of a neural cell and an endothelial cell.

7. The method of claim 6, wherein the neural cell is a motor neuron.

8. The method of claim 5, wherein the cell is a spinal cord cell.

9. The method of claim 1, further comprising allowing the angiogenin variant having a modified nuclear localization sequence to stimulate cell differentiation.

10. The method of claim 9, wherein an undifferentiated cell is stimulated to differentiate into a neural cell.

11. The method of claim 1, further comprising:
allowing nuclear translocation of the angiogenin variant having a modified nuclear localization sequence;
allowing the angiogenin variant having a modified nuclear localization sequence to stimulate ribosomal RNA transcription;
allowing ribosomal biogenesis;
allowing cell proliferation; and
allowing angiogenesis.

12. The method of claim 1, wherein the neurodegenerative disorder is selected from the group consisting of neuromuscular disorders, Alzheimer's disease, aphasia, Bell's palsy, Creutzfeldt-Jacob disease, cerebrovascular disease, encephalitis, epilepsy, prion disease, Huntington's disease, pain, phobia, movement disorders, Parkinson's disease, sleep disorders, Tourette syndrome, multiple sclerosis, neural tumors, neural autoimmune disorders, multiple sclerosis, pediatric neural disorders, autism, dyslexia, cerebral palsy, amyotrophic lateral sclerosis, Guillain-Barre syndrome, Charcot-Marie-Tooth disease, spinal muscular atrophy, muscular dystrophy, and spastic paraplegia.

13. The method of claim 1, wherein the administering is selected from the group consisting of intravenously administering, subcutaneously administering, intraperitoneally administering, intramuscularly administering, intrathecally administering and intraventricularly administering.

14. The method of claim 1, wherein the administering is intravenously administering.

15. The method of claim 1, wherein one or more symptoms of ALS are reduced in the subject.

16. The method of claim 15, wherein the one or more symptoms of ALS are selected from the group consisting of motor neuron degeneration, muscle weakness, muscle atrophy, motor neuron degeneration, fasciculation development, frontotemporal dementia, and premature death.

17. The method of claim 1, wherein the angiogenin variant having a modified nuclear localization sequence enters one or both of the brain and the spinal cord.

18. The method of claim 1, wherein one or both of muscle coordination and muscle function are improved in the subject.

19. The method of claim 1, wherein survival is prolonged in the subject.

20. The method of claim 1 wherein the modified nuclear localization sequence is a member selected from the group consisting of ³⁰KRRRG³⁴ (M30K) (SEQ ID NO:9), ³⁰RRRRG³⁴ (M30R) (SEQ ID NO:10), ³⁰MRRRK³⁴ (G34K) (SEQ ID NO:11), ³⁰MRRRR³⁴ (G34R) (SEQ ID NO:12), ³⁰KRRRR³⁴ (M30KG34R) (SEQ ID NO:13), ³⁰RRRRK³⁴ (M30RG34K) (SEQ ID NO:14), ³⁰RRRRR³⁴ (M30RG34R) (SEQ ID NO:15), and ³⁰KRRRK³⁴ (M30KG34K) (SEQ ID NO:16).

21. The method of claim 20 wherein the angiogenin variant further includes a D116H or Q117G substitution.

22. A method of therapeutically treating a neurodegenerative disorder in a subject in need thereof comprising:
administering to the subject a therapeutically effective amount of a composition comprising an isolated nucleic acid sequence encoding an angiogenin variant having a modified nuclear localization sequence;
expressing the angiogenin variant having a modified nuclear localization sequence in the subject;
allowing the angiogenin variant having a modified nuclear localization sequence to pass through one or both of the blood brain barrier and the blood spinal cord barrier; and
reducing one or more symptoms of the neurodegenerative disorder in the subject such that the neurodegenerative disorder is therapeutically treated.

23. The method of claim 22, wherein the nucleic acid sequence is administered using a gene therapy vector.

24. The method of claim 22, wherein the neurodegenerative disorder is ALS.

25. The method of claim 24, wherein one or more symptoms of ALS are reduced in the subject.

26. The method of claim 24, wherein the one or more symptoms of ALS are selected from the group consisting of motor neuron degeneration, muscle weakness, muscle atrophy, motor neuron degeneration, fasciculation development, frontotemporal dementia, and premature death.

27. The method of claim 22, wherein the angiogenin variant having a modified nuclear localization sequence enters one or both of the brain and the spinal cord.

28. The method of claim 22, wherein one or both of muscle coordination and muscle function are improved in the subject.

29. The method of claim 22, wherein survival is prolonged in the subject.

30. The method of claim 22 wherein the modified nuclear localization sequence is a member selected from the group consisting of ³⁰KRRRG³⁴ (M3 OK) (SEQ ID NO:9), ³⁰RRRRG³⁴ (M30R) (SEQ ID NO:10), ³⁰MRRRK³⁴ (G34K) (SEQ ID NO:11), ³⁰MRRRR³⁴ (G34R) (SEQ ID NO:12), ³⁰KRRRR³⁴ (M30KG34R) (SEQ ID NO:13), ³⁰RRRRK³⁴ (M30RG34K) (SEQ ID NO:14), ³⁰RRRRR³⁴ (M30RG34R) (SEQ ID NO:15), and ³⁰KRRRK³⁴ (M30KG34K) (SEQ ID NO:16).

31. The method of claim 30 wherein the angiogenin variant further includes a D116H or Q117G substitution.

32. A method of therapeutically treating a neurodegenerative disorder in a subject in need thereof comprising:
administering directly to the central nervous system of subject a therapeutically effective amount of a composition comprising an angiogenin variant having a modified nuclear localization sequence; and
reducing one or more symptoms of the neurodegenerative disorder in the subject such that the neurodegenerative disorder is therapeutically treated.

33. The method of claim 32, wherein direct delivery is performed using an infusion pump or a delivery scaffold.

34. The method of claim 32 wherein the modified nuclear localization sequence is a member selected from the group consisting of ³⁰KRRRG³⁴ (M30K) (SEQ ID NO:9), ³⁰RRRRG³⁴ (M30R) (SEQ ID NO:10), ³⁰MRRRK³⁴ (G34K) (SEQ ID NO:11), ³⁰MRRRR³⁴ (G34R) (SEQ ID NO:12), ³⁰KRRRR³⁴ (M30KG34R) (SEQ ID NO:13), ³⁰RRRRK³⁴ (M30RG34K) (SEQ ID NO:14), ³⁰RRRRR³⁴ (M30RG34R) (SEQ ID NO:15), and ³⁰KRRRK³⁴ (M30KG34K) (SEQ ID NO:16).

35. The method of claim 32 wherein the angiogenin variant further includes a D116H or Q117G substitution.

36. A method of therapeutically treating a neurodegenerative disorder in a subject in need thereof comprising:
administering directly to the central nervous system of subject a therapeutically effective amount of a composition comprising an isolated nucleic acid sequence encoding an angiogenin variant having a modified nuclear localization sequence;
expressing the angiogenin variant having a modified nuclear localization sequence in the subject; and
reducing one or more symptoms of the neurodegenerative disorder in the subject such that the neurodegenerative disorder is therapeutically treated.

37. The method of claim 36, wherein direct delivery is performed using an infusion pump or a delivery scaffold.

38. The method of claim 36 wherein the modified nuclear localization sequence is a member selected from the group consisting of ³⁰KRRRG³⁴ (M30K) (SEQ ID NO:9), ³⁰RRRRG³⁴ (M30R) (SEQ ID NO:10), ³⁰MRRRK³⁴ (G34K) (SEQ ID NO:11), ³⁰MRRRR³⁴ (G34R) (SEQ ID NO:12), ³⁰KRRRR³⁴ (M30KG34R) (SEQ ID NO:13), ³⁰RRRRK³⁴ (M30RG34K) (SEQ ID NO:14), ³⁰RRRRR³⁴ (M30RG34R) (SEQ ID NO:15), and ³⁰KRRRK³⁴ (M30KG34K) (SEQ ID NO:16).

39. The method of claim 38 wherein the angiogenin variant further includes a D116H or Q117G substitution.

40. Angiogenin of SEQ ID NO 1 having a nuclear localization sequence selected from the group consisting of ³⁰KRRRG³⁴ (M30K) (SEQ ID NO:9), ³⁰RRRRG³⁴ (M30R) (SEQ ID NO:10), ³⁰MRRRK³⁴ (G34K) (SEQ ID NO:11), ³⁰MRRRR³⁴ (G34R) (SEQ ID NO:12), ³⁰KRRRR³⁴ (M30KG34R) (SEQ ID NO:13), ³⁰RRRRK³⁴ (M30RG34K) (SEQ ID NO:14), ³⁰RRRRR³⁴ (M30RG34R) (SEQ ID NO:15), and ³⁰KRRRK³⁴ (M30KG34K) (SEQ ID NO:16).

41. The angiogenin of claim 40 having a D116H or Q117G substitution.


SEQUENCE LISTING (16 sequences)

SEQ ID NO: 1 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 2 - protein, 147 aa, Pan troglodytes
MVMGLGVLLLVFVLGLGLTPPTLAQDNSRYTHFLTQHYDAKPQGRDHRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 3 - protein, 147 aa, Gorilla gorilla; identical to SEQ ID NO: 1

SEQ ID NO: 4 - protein, 146 aa, Mus musculus
MAISPGPLFLIFVLGLGLTPPTLAQDDSRYTKFLTQHHDAKPKGRDDRYCERMMKRRSLTSPCKDVNTFIHGNKCNIKAICEGANGSPYRENLRMSKSPFQVTTCKHTGGSPRPPCQYRASAGFRHVVIACENGLPVHLDESFFSL

SEQ ID NO: 5 - protein, 146 aa, Rattus norvegicus
MEMSLRPLLLVFVLGLVSTPSTLAQDDPRYTKFLTQHYDAKPKGRDARYCESMMRRRGLTSPCKEVNTFIHGNKGSIKAICEGANGSPYGENLRISQSPFQITTCKHTGGSPRPPCRYRASAGFRHVVIACENGLPVHFDESFISL

SEQ ID NO: 6 - protein, 148 aa, Bos primigenius
MVMVLSPLLLVFILGLGLTPVAPAQDDYRYIHFLTQHYDAKPKGRNDEYCFNNMKNRRLTRPCKDRNTFIHGNKNDIKAICEDRNGGPYRGDLRISKSEFQITICKHKGGSSRPPCRYGATEDSRVIVVGCENGLPVHFDESFITPRH

SEQ ID NO: 7 - protein, 148 aa, Sus domestica
MVILLGPLLLVFMLGLGLAPLSLAKDEDRYTHFLTQHYDAKPKGRDGRYCESIMKQRGLTRPCKEVNTFIHGTRNDIKAICEDKNGEPYNENFRRSKSPFQITTCKHKGGSNRPPCGYRATAGFRTIAVACENGLPVHFDESFIITSQ

SEQ ID NO: 8 - protein, 7 aa, Artificial Sequence
  nuclear localization signal
IMRRRGL

SEQ ID NO: 9 - protein, 5 aa, Artificial Sequence
  nuclear localization signal
KRRRG

SEQ ID NO: 10 - protein, 5 aa, Artificial Sequence
  nuclear localization signal
RRRRG

SEQ ID NO: 11 - protein, 5 aa, Artificial Sequence
  nuclear localization signal
MRRRK

SEQ ID NO: 12 - protein, 5 aa, Artificial Sequence
  nuclear localization signal
MRRRR

SEQ ID NO: 13 - protein, 5 aa, Artificial Sequence
  nuclear localization signal
KRRRR

SEQ ID NO: 14 - protein, 5 aa, Artificial Sequence
  nuclear localization signal
RRRRK

SEQ ID NO: 15 - protein, 5 aa, Artificial Sequence
  nuclear localization signal
RRRRR

SEQ ID NO: 16 - protein, 5 aa, Artificial Sequence
  nuclear localization signal
KRRRK